(19) United States
(12) Patent Application Publication
(10) Pub. No.: US 2014/0323819 A1
(43) Pub. Date: Oct. 30, 2014

(54) MULTI-PARAMETER TEST UNITS FOR INITIAL INDICATION OF MEDICAL SYMPTOMS

(71) Applicant: Elwha LLC, Bellevue, WA (US)

(72) Inventors: Roderick A. Hyde, Redmond, WA (US); Wayne R. Kindsvogel, Seattle, WA (US); Gary L. McKnight, Bothell, WA (US)

(73) Assignee: Elwha LLC, a limited liability company of the State of Delaware

(21) Appl. No.: 13/872,578

(22) Filed: Apr. 29, 2013

(51) Int. Cl. A61B 5/00; A61B 5/01

(52) U.S. Cl. CPC A61B 5/682; A61B 5/01
USPC 600/301

(57) ABSTRACT

Disposable test units to assist medical personnel are described. In some embodiments, a test unit includes: a solid support; a cavity in the solid support; a reaction chamber internal to the solid support, including a plurality of biochemical reagents; a first internal channel in the solid support, connected at a first end to the cavity, connected at a second end to the reaction chamber; at least one pathogen detection region internal to the solid support; a second internal channel in the solid support, connected at a first end to the reaction chamber, connected at a second end to the at least one pathogen detection region; a temperature detector; and a persistent visible temperature indicator attached to the temperature detector.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

None.

If the listings of applications provided above are inconsistent with the listings provided via an ADS, it is the intent of the Applicant to claim priority to each application that appears in the Priority Applications section of the ADS and to each application that appears in the Priority Applications section of this application.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

In some embodiments, a test unit includes: a solid support including a surface configured to reversibly mate with a skin surface of an individual; a cavity in the solid support; a reaction chamber internal to the solid support, the reaction chamber including a plurality of detection agents within a space; a first internal channel in the solid support, the first internal channel connected at a first end to the cavity, the first internal channel connected at a second end to the reaction chamber; at least one pathogen detection region internal to the solid support, each of the at least one pathogen detection region including a visual indicator of one or more of the plurality of detection agents; a second internal channel in the solid support, the second internal channel connected at a first end to the reaction chamber, the second internal channel connected at a second end to the at least one pathogen detection region; a temperature detector affixed to the solid support; and a persistent visual temperature indicator attached to the temperature detector.

In some embodiments, a test unit includes: a solid support including a surface configured to reversibly mate with the skin of an individual; a nasal fluid receiving cavity in the solid support; a reaction chamber internal to the solid support, the reaction chamber includes a plurality of detection agents within a space; a first internal channel to the solid support, the first internal channel including a first end attached to the nasal fluid receiving cavity, the first internal channel including a second end attached to the reaction chamber; at least one pathogen detection region internal to the solid support, each of the at least one pathogen detection region including a visual indicator of one or more of the plurality of detection agents; at least one second internal channel to the solid support, the at least one second internal channel including a first end attached to the at least one reaction chamber, the at least one second internal channel including a second end attached to the at least one pathogen detection region; a temperature detector affixed to the solid support; and a persistent visual temperature indicator attached to the temperature detector.

In some embodiments, a test unit includes: a solid support including a region configured to be enclosed within an oral cavity of an individual; an oral fluid receiving cavity in the solid support; a reaction chamber internal to the solid support, the reaction chamber including a plurality of detection agents within a space; a first internal channel to the solid support, the first internal channel including a first end attached to the oral fluid receiving cavity, the first internal channel including a second end attached to the reaction chamber; at least one pathogen detection region internal to the solid support, each of the at least one pathogen detection region including a pathogen detector and a visual indicator; at least one second internal channel to the solid support, the at least one second internal channel including a first end attached to the at least one reaction chamber, the at least one second internal channel including a second end attached to the at least one pathogen detection region; a temperature detector affixed to the solid support; and a persistent visual temperature indicator attached to the temperature detector.

In some embodiments, a test unit includes: a solid support including a surface configured to reversibly mate with a skin surface of an individual; a cavity in the solid support; at least one pathogen detection region internal to the solid support, each of the at least one pathogen detection region including an electronic detector of one or more of the plurality of detection agents in combination with an analyte; an internal channel in the solid support, the internal channel connected at a first end to the cavity, the first channel connected at a second end to the at least one pathogen detection region; an electronic temperature detector affixed to the solid support; a processor attached to both the electronic detector and the electronic temperature detector; and a persistent visible indicator attached to the processor.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a schematic of a medical caregiver evaluating a series of individuals for potential infection.

FIG. 2 illustrates an embodiment of a test unit.

FIG. 3 depicts an embodiment of a test unit after use.

FIG. 4 shows a test unit from a cross-section viewpoint.

FIG. 5 illustrates a test unit from a cross-section viewpoint.

FIG. 6 depicts an embodiment of a test unit.

FIG. 7 shows an embodiment of a test unit after use.

FIG. 8 illustrates an embodiment of a test unit.

FIG. 9 depicts an embodiment of test unit configured for oral use.

FIG. 10 shows an embodiment of test unit configured for oral use after use.

FIG. 11 illustrates an embodiment of a test unit.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Test units described herein can be utilized for rapid screening of individuals as an easy, cost-effective testing system for an initial, multi-parameter screen for infection. The test units described provide healthcare personnel with information regarding several parameters regarding an individual's health on a single test unit. Test units described herein can be easily utilized by healthcare providers to provide results regarding several medical parameters relating to infection in a single test unit. The results from the test units can be utilized by healthcare personnel to mitigate further infection risk in a patient population and as a basis for quick intervention for infected individuals. The test units provide persistent results and can be administered by a first healthcare provider and evaluated, or re-evaluated, by a second healthcare provider after the test unit is administered to a patient. The test units provide persistent results that can be visually recorded, such as through a photograph or scan, for addition to a patient's medical record. The test units provide a consistent screening method that can be used on a regular basis, such as daily or weekly, in periods of high infection risk (e.g. “flu season”). The results from the test unit can provide specific information about the presence of pathogens, which can have clinical utility to healthcare providers. See: Fleming, “Influenza Diagnosis and Treatment: A View From Clinical Practice,” Phil. Trans. R. Soc. Land. B 356: 1933-1943 (2001), which is incorporated by reference herein. Rapid identification of influenza diagnosis, for example, can assist medical personnel to manage high-risk contacts of patients, prescribe antivirals as needed, and avoid unnecessary antibiotic use by patients. See, e.g., Woolpert et al., “Determination of Clinical and Demographic Predictors of Laboratory-Confirmed Influenza with Subtype Analysis,” BMC Infectious Diseases 12: 129, (2012) and Michiels et al., “Clinical Predication Rules Combining Signs, Symptoms and Epidemiological Context to Distinguish Influenza from Influenza-like Illnesses in Primary Care: A Cross Sectional Study,” BMC Family Practice 12:4, (2011), which are each incorporated herein by reference. For example, it has been shown that evaluation of multiple parameters provides an accurate clinical decision rules for diagnosis of influenza. See, e.g., Ebell et al., “Development and validation of a Clinical Decision Rule for the Diagnosis of Influenza,” JABFM 25 (1) 55-62 (2011), which is incorporated by reference.

For example, in some embodiments, multi-parameter test units can be utilized by healthcare workers at a nursing home as a routine, potentially daily, screening tool of patients in the nursing home for fever and infection during an influenza epidemic. A low cost, disposable test unit can be used to measure each patient's body temperature and to detect viral infections in their nasal fluids. A low cost, disposable test unit can be used to measure each patient's body temperature and to detect viral infections in their oral fluids. In some embodiments, the multi-parameter test unit incorporates a chemical thermometer and lateral flow immunoassays in a single device that provides a rapid visual readout of each patient's results. In some embodiments, the multi-parameter test unit incorporates an electronic thermometer and electronically-detectable assays for pathogens and infection symptoms in a single device that provides a rapid visual readout of each patient's results. The test unit provides persistent visual results, so the test strip can be administered by a first healthcare worker and evaluated by one or more subsequent healthcare workers. A test strip can also be documented into a medical record through a visual scan or a photograph. In some embodiments, a test strip includes electronic transmission components configured to transmit test results to a medical record system.

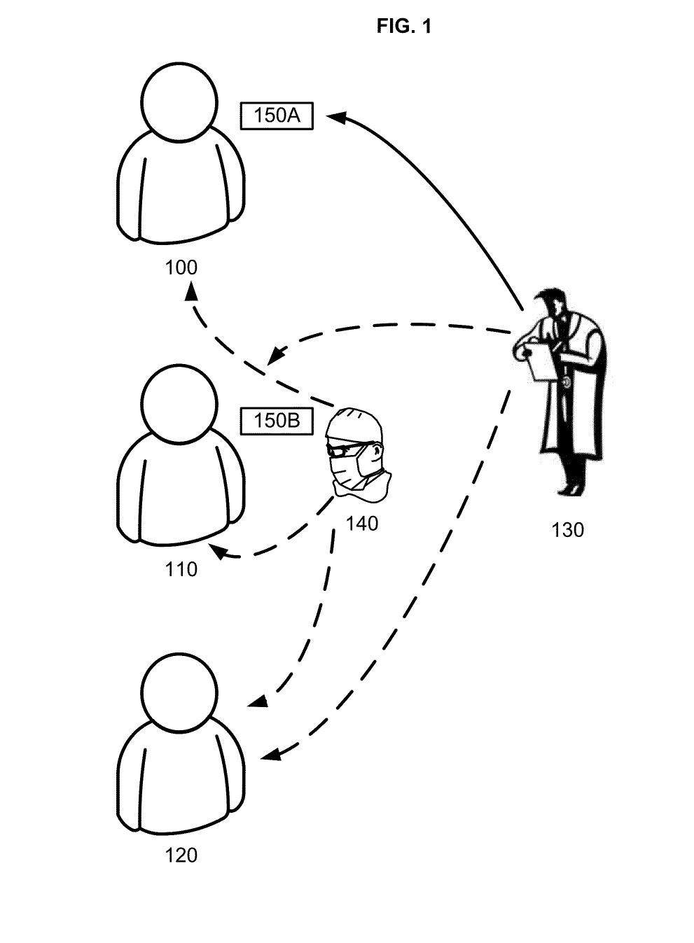

FIG. 1 illustrates aspects of possible use of multi-parameter test units, as described herein. A first patient 100 is administered a first test unit 150A by a first medical professional, 140. The first medical professional 140 then proceeds to administer a second test unit 150B to a second patient 110. After administration of test units 150A, 150B to the first patient 100 and the second patient 110, the first medical professional 140 continues to administer a test unit to a third patient 120, and possibly subsequent patients. The first test unit 150A and the second test unit 150B are left with the respective patients or placed in a central location for evaluation. A second healthcare professional 130 can subsequently read and evaluate the first and second test units 150A, 150B. The second healthcare professional 130 can also document the test units, for example by scan or photograph included into a digital health record. Since each test unit includes persistent visual indicators of the results of the included assays, a second healthcare professional can evaluate the test results at a time after the tests are administered, which may assist with healthcare personnel time management.

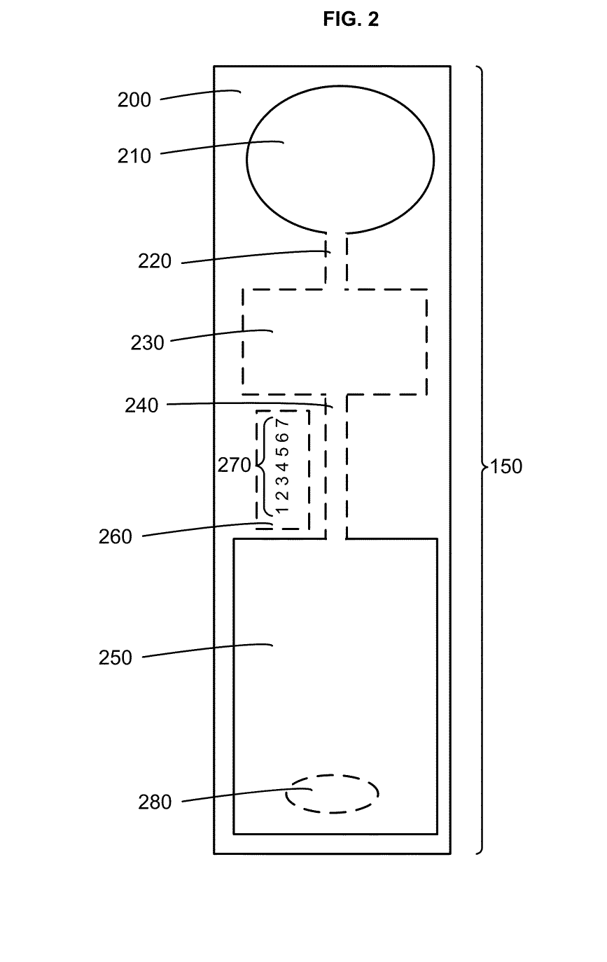

FIG. 2 illustrates an embodiment of a test unit. The test unit 150 includes a solid support 200 including a surface configured to reversibly mate with a skin surface of an individual. The test unit 150 also includes a cavity 210 in the solid support 200. The test unit 150 includes a reaction chamber 230 internal to the solid support 200, the reaction chamber 230 including a plurality of detection agents within a space. Although the reaction chamber 230 is internal to the solid support 200 and, therefore, not externally visible, its approximate location within the solid support 230 is shown as dashed lines in FIG. 2. The test unit 150 includes a first internal channel 220 in the solid support 200, the first internal channel 220 connected at a first end to the cavity 210, the first internal channel 220 connected at a second end to the reaction chamber 230. Although the first internal channel 220 is internal to the solid support 200 and, therefore, not externally visible, its approximate location within the solid support 230 is shown as dashed lines in FIG. 2. The test unit 150 includes at least one pathogen detection region 250 internal to the solid support 200, each of the at least one pathogen detection region 250 including a visual indicator of one or more of the plurality of detection agents. The visual indicators of the pathogen detection region 250 are visible externally to the test unit 150. For example, in some embodiments the visible indicators include color changes caused by biochemical reactions, which are visible through one or more translucent layers of the solid support 200. See, e.g. U.S. Pat. No. 5,053,339 “Color Changing Device for Monitoring Shelf-Life of a Perishable Products,” to Patel, and U.S. Pat. No. 5,667,303, “Time-Temperature Integrating Indicator Device,” to Arens et al., which are each incorporated by reference. The pathogen detection region 250 can include a visual indicator of a positive control protein 280 to verify assay results. Although the visual indicator of a positive control protein 280 is not active in FIG. 2 (e.g. the test unit has not been used to activate the positive control indicator), the region where the visual indicator of a positive control protein 280 would be visible on a used test unit is marked with a dotted line. The test unit 150 includes a second internal channel 240 in the solid support 200, the second internal channel 240 connected at a first end to the reaction chamber 230, the second internal channel 240 connected at a second end to the at least one pathogen detection region 250. Although the second internal channel 240 is internal to the solid support 200 and, therefore, not externally visible, its location within the solid support 230 is shown as dashed lines in FIG. 2. The test unit 150 includes a temperature detector 260 affixed to the solid support 200, and a persistent visual temperature indicator 270 attached to the temperature detector 260. In the embodiment illustrated, the temperature detector 260 is affixed to the solid support at a position adjacent to the face of the solid support 200 not visible in FIG. 2, but its approximate location is shown as dashed lines in FIG. 2.

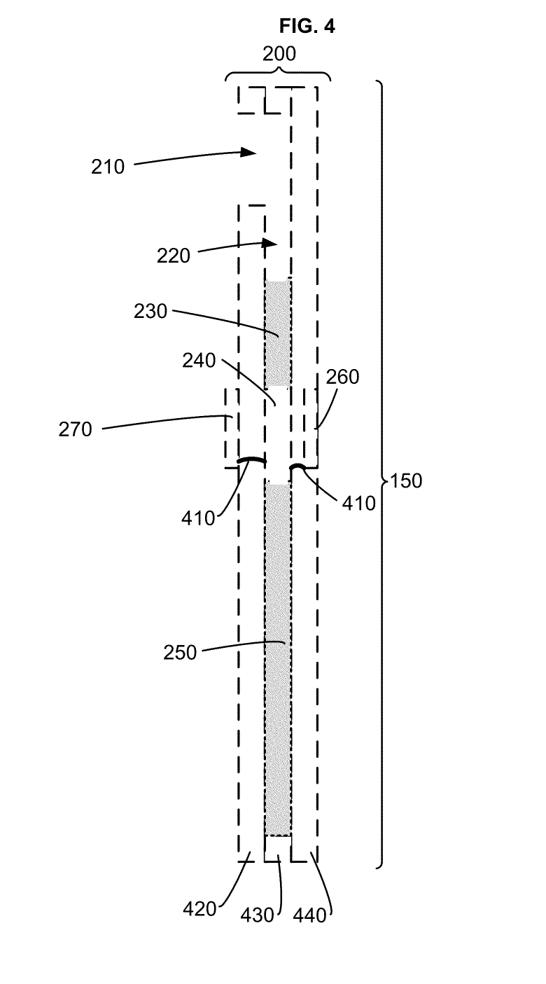

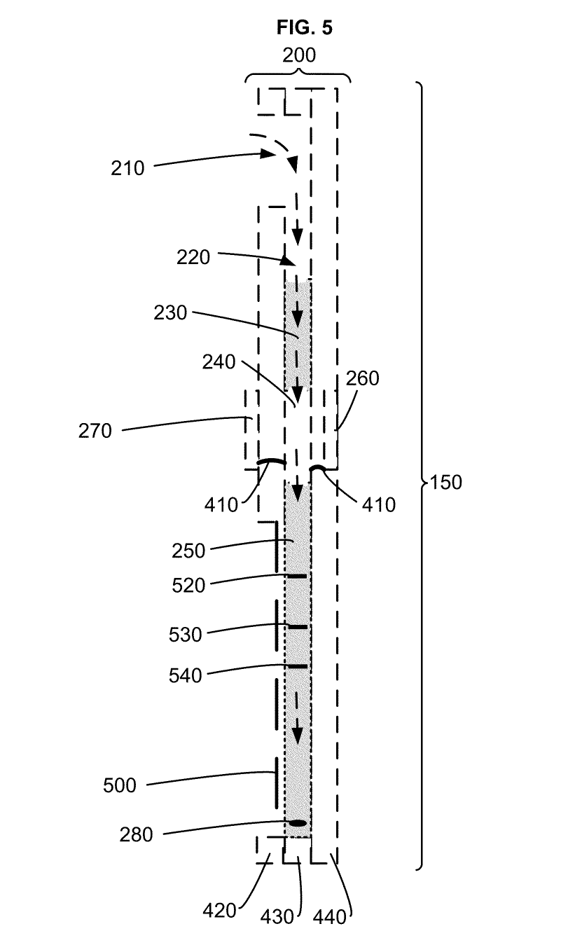

The test unit 150 shown in the embodiment illustrated in FIG. 2 includes a solid support 200 including a surface configured to reversibly mate with a skin surface of an individual. For example, in some embodiments a solid support 200 includes a substantially flat solid support. For example, in some embodiments a solid support 200 includes a substantially flexible solid support, the solid support having sufficient flexibility to reversibly mate with a skin surface of an individual at an intended location on the individual's body. For example, in some embodiments a solid support 200 includes a substantially flexible solid support, the solid support having sufficient flexibility to reversibly mate with a forehead surface of an individual. In some embodiments a solid support 200 includes a substantially flexible solid support, the solid support having sufficient flexibility to reversibly mate with a wrist surface of an individual. In some embodiments, a solid support 200 includes a paper-based solid support. For example, a solid support can include a paper-based solid support with sufficient strength and flexibility to support the other components of the test unit in the particular embodiment. In some embodiments, a solid support 200 includes a plastic-based solid support. For example, a solid support can include a plastic-based solid support with sufficient strength and flexibility to support the other components of the test unit in the particular embodiment. In some embodiments, a solid support includes both a paper-based component and a plastic-based component, for example positioned as layers (see, e.g. FIGS. 4 and 5). In some embodiments, a solid support includes a plurality of layers combined to form the solid support structure. In some embodiments, a solid support includes a surface configured for placement adjacent to the skin, and at least one bio-compatible adhesive on the surface configured for placement adjacent to the skin. In some embodiments, the test unit can include skin-compatible adhesives on the test unit surface that reversibly mates with the surface of an individual's skin to hold the test unit in place on the skin surface while a temperature measurement is taken. The test unit is designed to be single-use and disposable, so the solid support should be fabricated from a disposable and relatively inexpensive material.

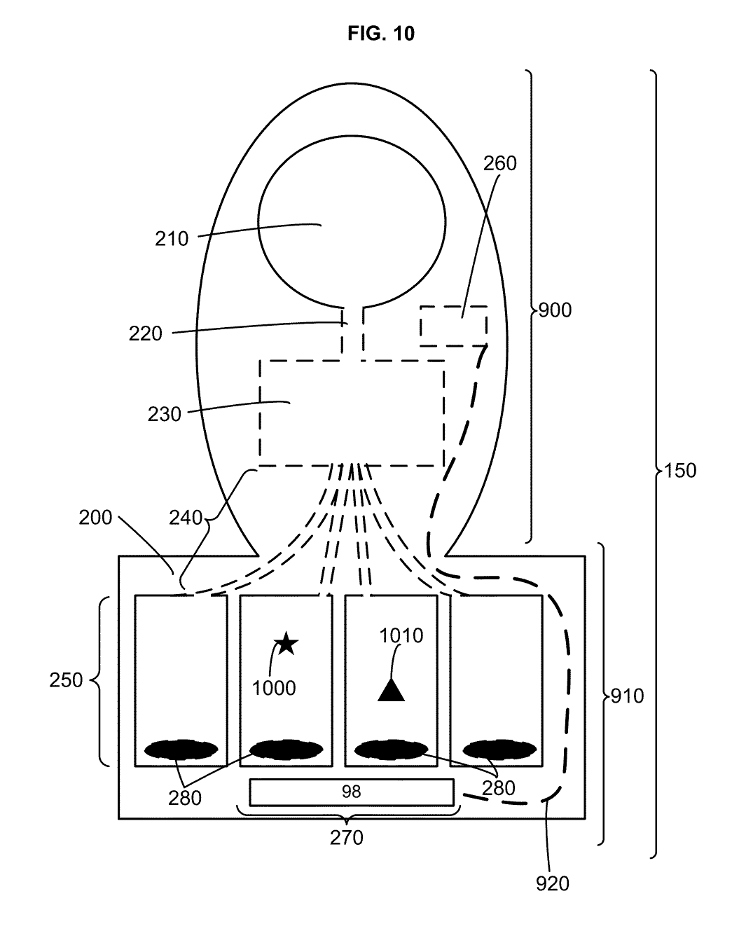

Some embodiments include a solid support including a region configured to be enclosed within an oral cavity of an individual. See, e.g. FIG. 10. Some embodiments include a solid support including a region configured to be partially enclosed within an oral cavity of an individual. For example, a test unit includes, in some embodiments, a solid support including a region configured to be enclosed within the oral cavity of an individual, including: a first region configured to fit within the oral cavity; and a second region configured to be positioned outside the oral cavity.

As shown in FIG. 2, the test unit 150 includes a cavity 210 in the solid support 200. The cavity 210 is of a size and shape to receive a body fluid from an individual in a quantity sufficient for the immunoassay section of the test unit to be operational. The size and shape of the cavity 210 is, therefore, dependent on factors including the body fluid that the test unit 150 is configured to analyze, and the origin of the body fluid. The size and shape of the cavity 210 is also dependent on the volume of body fluid required for the immunoassay(s) on the test unit. For example, the size and shape of the cavity can include an indentation in a surface of the solid support, the indentation configured to retain no more than approximately 1 milliliter of body fluid. For example, the size and shape of the cavity can include an indentation in a surface of the solid support, the indentation configured to retain no more than approximately 100 microliters of body fluid. For example, the size and shape of the cavity can have an equivalent volume to the volume of the interior of the reaction chamber and the pathogen detection region. For example, the size and shape of the cavity can have a volume greater than the volume of the interior of the reaction chamber and the pathogen detection region. In some embodiments, the cavity is of a size and shape to directly contact the body part of an individual and to receive a body fluid from the body part. The cavity can be fabricated by removal of a section of the solid support material to form an indentation during fabrication of the test unit. The cavity can be fabricated by gaps or holes within some layers of a plurality of layers forming the test unit. For example, in some embodiments the cavity includes an indentation in a surface of the solid support. For example, in some embodiments the cavity includes an indentation in a surface of the solid support, the indentation configured to retain a body fluid. For example, the cavity can include an indentation configured to retain a particular body fluid through capillary action to the sides of the cavity. For example, the cavity can include one or more grooves or channels configured to retain a particular body fluid through capillary action.

For example, in some embodiments the test unit is configured to analyze proteins present in nasal fluid. In some embodiments, a test unit includes a nasal fluid receiving cavity in the solid support. In some embodiments, a test unit includes an indentation in a surface of the solid support, the indentation configured to retain nasal fluid directly from an nasal cavity of an individual. The size and shape of the cavity in a test strip configured to analyze proteins present in nasal fluid is large enough to receive a sufficient quantity of nasal fluid from an individual for use in the immunoassay(s) present on the test unit. In some embodiments, a test unit includes a nasal fluid receiving cavity including an indentation in a surface of the solid support, the indentation configured to retain no more than approximately 1 milliliter of nasal fluid. In some embodiments, a test unit includes an indentation in a surface of the solid support, the indentation configured to retain no more than approximately 100 microliters of nasal fluid. For example, the shape and size of the cavity should hold sufficient nasal fluid to substantially mix with the detection agents present in the reaction chamber. In some embodiments, the volume of the interior of the cavity is larger than the volume of the space within the reaction chamber. In some embodiments, the volume of the interior of the cavity can have a volume greater than the volume of the interior of the reaction chamber and the pathogen detection region. In some embodiments, the cavity is of a size and shape to receive nasal fluid directly from an individual's nose. For example, the cavity can receive nasal fluid when the individual wipes his or her nose across the cavity within the test unit. For example, the cavity can receive nasal fluid when the individual exhales through his or her nostrils in against the cavity within the test unit.

For example, in some embodiments the test unit is configured to analyze proteins present in oral fluid. See: Miller et al., “Current Developments in Salivary Diagnostics,” Biomark Med. 4(1): 171-189 (2010), which is incorporated by reference herein. The test unit can be configured to receive oral fluid from an individual, detect specific proteins present in the oral fluid, and change a visible indicator in response to the detection. For example, in some embodiments a test unit includes an oral fluid receiving cavity in the solid support including an indentation in a surface of the solid support, the indentation configured to receive and retain oral fluid directly from an oral cavity. The size and shape of the cavity in a test strip configured to analyze proteins present in oral fluid is large enough to receive a sufficient quantity of oral fluid from an individual for use in the immunoassay(s) present on the test unit. For example, in some embodiments a test unit includes an oral fluid receiving cavity in the solid support including an indentation in a surface of the solid support, the indentation configured to retain no more than approximately 1 milliliter of oral fluid. For example, in some embodiments a test unit includes an oral fluid receiving cavity in the solid support including an indentation in a surface of the solid support, the indentation configured to retain no more than approximately 100 microliters of oral fluid. For example, the shape and size of the cavity should hold sufficient oral fluid to substantially mix with the detection agents present in the reaction chamber. In some embodiments, the volume of the interior of the cavity is larger than the volume of the space within the reaction chamber. In some embodiments, the volume of the interior of the cavity can have a volume greater than the volume of the interior of the reaction chamber and the pathogen detection region. In some embodiments, the cavity is of a size and shape to receive oral fluid directly from an individual's mouth. For example, the cavity can receive oral fluid when the individual licks or spits into the cavity within the test unit. For example, the cavity can receive oral fluid when the individual holds the test unit within his or her mouth.

The test unit shown in FIG. 2 includes a reaction chamber 230 internal to the solid support, the reaction chamber including a plurality of detection agents within a space. The space of the reaction chamber should be of a sufficient size to contain the detection agents along with sufficient body fluid to mix with the detection agents to activate the visual indicators in the pathogen detection region. For example, in some embodiment the space within the reaction chamber has an approximate volume of 500 microliters. For example, in some embodiment the space within the reaction chamber has an approximate volume of 400 microliters. For example, in some embodiment the space within the reaction chamber has an approximate volume of 300 microliters. For example, in some embodiment the space within the reaction chamber has an approximate volume of 200 microliters. For example, in some embodiment the space within the reaction chamber has an approximate volume of 100 microliters. In some embodiments, the volume of the space within the reaction chamber is approximately equivalent to the volume of the interior of the at least one pathogen detection region internal to the solid support. In some embodiments, the volume of the space within the reaction chamber is larger than the volume of the interior of the at least one pathogen detection region internal to the solid support. For example, in some embodiments, the volume of the space within the reaction chamber is at least 10% greater than the volume of the interior of the at least one pathogen detection region internal to the solid support. For example, in some embodiments, the volume of the space within the reaction chamber is at least 20% greater than the volume of the interior of the at least one pathogen detection region internal to the solid support. In some embodiments, the interior surface of the reaction chamber, adjacent to the interior space, includes a surface configured to stabilize the plurality of detection agents within the space during storage and shipment of the test unit prior to use.

The reaction chamber of a test unit includes a plurality of detection agents within a space. The detection units selected for a specific embodiment depend on the particular pathogen(s) and pathogen protein(s) that the test unit is configured to detect. In some embodiments, the plurality of detection agents include one or more detection agents including an antibody configured to bind with a pathogen protein. In some embodiments, the plurality of detection agents include monoclonal antibodies (MAbs) conjugated with colloidal gold particles which are known to bind to a specific pathogen protein. For example, in some embodiments the plurality of detection agents include monoclonal antibodies (MAbs) known to bind to proteins from all known strains of influenza type A and/or influenza type B conjugated with colloidal gold particles. See: U.S. Pat. No. 8,163,474, “NS1-NP Diagnostics of Influenza Virus Infection,” to Lu et al.; U.S. Pat. No. 7,595,151, “Methods and Compositions for Diagnosis and Treatment of Influenza,” to Lu et al.; U.S. Pat. No. 7,595,152, “Detection of Influenza Virus,” to Lu et al.; U.S. Patent Application Publication Nos. 2007/0161078 and 2010/0112547, “Methods and Compositions for Diagnosis and Treatment of Influenza,” to Lu et al.; U.S. Patent Application Publication Nos. 2007/0224594 and 2010/0092944, “Detection of Influenza Virus,” to Lu et al.; U.S. Patent Application Publication No. 2009/0280504, “NS1-NP Diagnostics of Influenza Virus Infection,” to Lu et al.; U.S. Patent Application Publication No. 2010/0028855, “Detection of Influenza Virus Type B,” to Lu et al.; U.S. Patent Application Publication No. 2012/0258890, “NS1-NP Diagnostics of Influenza Virus Infection,” to Lu et al.; and U.S. Patent Application Publication Nos. 2010/0143884, and 2011/0027775, “Detection of Influenza Virus,” to Lu et al., which are each incorporated herein by reference. In some embodiments, the plurality of detection agents include one or more detection agents including an enzyme.

FIG. 2 illustrates that a first internal channel 220 is present within the solid support 200, the first internal channel 220 connected at a first end to the cavity 210, the first internal channel 220 connected at a second end to the reaction chamber 230. FIG. 2 also illustrates a second internal channel 240 in the solid support 200, the second internal channel 240 connected at a first end to the reaction chamber 230, the second internal channel 240 connected at a second end to the at least one pathogen detection region 250. Although the first internal channel 220 and the second internal channel 240 are both internal to the solid support 200 and, therefore, not externally visible, the approximate location of the first internal channel 220 and the second internal channel 240 within the solid support 200 are illustrated in FIG. 2 with dotted lines. In some embodiments, the first internal channel includes a fluid-control film component, the fluid-control film oriented to permit directional flow of fluid from the cavity to the reaction chamber. In some embodiments, the second internal channel includes a fluid-control film component, the fluid-control film oriented to permit directional flow of fluid from the reaction chamber to the at least one pathogen detection region. See U.S. Pat. No. 6,420,622 to Johnston et al., “Medical Article Having Fluid Control Film,” which is incorporated by reference.

The test unit 150 illustrated in FIG. 2 includes at least one pathogen detection region 250 internal to the solid support 200, each of the at least one pathogen detection region 250 including a visual indicator of one or more of the plurality of detection agents. For example, in some embodiments a test unit includes reagents to carry out an immunoassay within the test unit, and the visual indicator is a chemical indicator of the immunoassay results. For example, in some embodiments, a pathogen detection region includes a membrane, and a plurality of capture agents affixed to the membrane in a pattern. For example, a pathogen detection region can include a nitrocellulose membrane with capture agents including MAbs immobilized on the membrane surface. For example, in some embodiments a pathogen detection region can include a nitrocellulose membrane with capture agents including MAbs that specifically bind to one or more influenza-specific proteins. The MAbs can be immobilized on the nitrocellulose membrane in, for example, a pixilated pattern that allows independent flow, capture and detection of antigens at each point in the pattern. Lateral flow assays to detect antigens in multiplex are described (see e.g., U.S. Patent Application Publication No. 2012/0184462, “Lateral Flow Assays Using Two Dimensional Features,” to O'Farrell et al., and U.S. Patent Application Publication No. 2010/0143884, “Detection of Influenza Virus,” to Lu et al., which are each incorporated herein by reference). The lateral flow assays can be evaluated by visual inspection of the spot pattern on the lateral flow assay section of the test unit. After an immunoassay is carried out with the body fluid of a patient, the pattern of color change on the membrane serves as a visible indicator to a medical professional regarding the results of the assay. For example, a particular pattern of color changes may indicate a positive result for influenza A virus particles. For example, a particular pattern of color changes may indicate a positive result for influenza B virus particles. In some embodiments, a test unit includes a plurality of pathogen detection regions. For example, a test unit can include a plurality of pathogen detection regions arranged in parallel on the test unit. After an immunoassay is carried out with the body fluid of a patient, the pattern of color change on each of the plurality of pathogen detection regions on a single test unit serves as a visible indicator to a medical professional regarding the results of the assay. For example, a positive color change in the first pathogen detection region can indicate a positive result for influenza A proteins. For example, a positive color change in the second pathogen detection region can indicate a positive result for influenza B proteins. In some embodiments, a test unit includes at least one control visual indicator in one or more of the pathogen detection regions on a single test unit. For example, the control visible indicator can include an indicator of the presence of a control protein, such as a protein normally present in saliva or nasal fluid. A positive result for the control visual indicator in the pathogen detection region on a test unit can demonstrate to a medical professional that the test unit operated properly, even if no other visible indicator indicated a positive result with a particular sample.

For a specific embodiment of a test unit, the detection agents within the reaction chamber of the test unit and the visible indicators of the detection agents within the pathogen detection region are selected to indicate the presence of pathogens that could be present in a particular body fluid from a patient. In some embodiments of a test unit, the plurality of detection agents within the reaction chamber include detection MAbs labeled with colloidal gold, and the visible indicators of one or more of the plurality of detection agents included in a pathogen detection region includes bound capture MAbs affixed to a nitrocellulose membrane. For example, the detection MAbs labeled with colloidal gold can be configured to bind to one or more influenza proteins, and the bound capture MAbs within the pathogen detection region can bind specifically to the detection MAbs labeled with colloidal gold. The colloidal gold particles will form a visible indicator in the pathogen detection region after they are immobilized at a specific location by the bound capture MAbs at that location. In some embodiments, the labeled detection MAbs within the reaction chamber include MAbs specific for pathogen proteins such as viral pathogens or bacterial pathogens. For example, one or more labeled detection MAbs can include MAbs specific to one or more of: influenza A, influenza B, respiratory syncytial virus, adenovirus, parainfluenza virus, Streptococcus pneumoniae, Neisseria meningitidis and Mycoplasma. In some embodiments, the labeled detection MAbs within the reaction chamber include MAbs specific for proteins associated with inflammation from the patient. For example, one or more labeled detection MAbs can include MAbs specific to one or more of: C-reactive protein (CRP), interleukin 1-β, and β-glucuronidase.

As shown in FIG. 2, in some embodiments a test unit includes a temperature detector 260 affixed to the solid support 200, and a persistent visible temperature indicator 270 attached to the temperature detector 260. For example, in some embodiments a temperature detector can include an electronic temperature detector configured to measure temperature in the physiological range at a location adjacent to the surface of the solid support configured to reversibly mate with the skin surface of an individual, and a persistent visible temperature indicator attached to the electronic temperature detector that maintains visibility over time. For example, a persistent visible temperature indicator attached to an electronic temperature detector can include a persistent electronic indicator. For example, a persistent visible temperature indicator attached to an electronic temperature detector can include an e-ink device. In embodiments including an electronic temperature detector, the test unit can include an attached power source, such as a battery. For example, in some embodiments a temperature detector can include a chemical temperature detector that includes a persistent visible temperature indicator integrated with the chemical temperature detector. For example, a persistent visible temperature indicator attached to a chemical temperature detector can include a persistent chemical indicator. In some embodiments, a chemical temperature detector includes a persistent visible temperature indicator by a change in appearance when going from solid to liquid at a specific temperature. See e.g., U.S. Pat. Nos. 4,232,552, 4,339,207 and 4,362,645, each titled “Temperature Indicating Compositions of Matter,” to Hof et al., which are each incorporated herein by reference. See U.S. Pat. No. 5,816,707, “Reversible Chemical Thermometer,” to Hof, which is incorporated by reference. In some embodiments, a chemical temperature detector includes a persistent visible temperature indicator that includes dyes which become visible after a phase change occurs. For example, chemical thermometers including dye-based persistent visible temperature indicators adapted for measuring temperature with an accuracy of approximately 0.2° F. are described. See e.g., U.S. Pat. No. 5,401,100 issued to Thackston et al. titled “Axillary Thermometer Packaging,” which is incorporated herein by reference.

In some embodiments, a heat conducting unit can be affixed to the surface of the solid support configured to reversibly mate with the skin surface at a position adjacent to the temperature detector, the heat conducting unit positioned to enhance thermal conduct between the skin surface and the temperature detector. For example, an aluminum heat conducting unit can be in a position adjacent to one face of a series of wells including chemical temperature detectors and indicators, the aluminum heat conducting unit positioned to efficiently transfer heat from the body to the temperature indicator wells when the test unit contacts the skin. In some embodiments, a test unit includes a thin plastic heat conducting unit on the surface of the solid support configured to reversibly mate with the skin surface in a position adjacent to one face of the temperature detector, the heat conducting unit positioned to efficiently transfer heat from the body to the temperature detector when the test unit is in contact with the skin.

In some embodiments, a test unit includes a processor attached to both the at least one pathogen detection region internal to the solid support and to the temperature detector affixed to the solid support. The processor is connected to both the pathogen detection region and to the temperature detector so that the processor is configured to accept information from both the pathogen detection region and the temperature detector. The processor can include, for example, a look-up table stored in memory, the look-up table including values for information from either or both the pathogen detection region and the temperature detector that are identified as diagnostically “positive” or “negative.” For example, it has been shown that evaluation of multiple parameters provides an accurate clinical decision rules for diagnosis of influenza. See, e.g., Ebell et al., “Development and validation of a Clinical Decision Rule for the Diagnosis of Influenza,”JABFM 25 (1) 55-62 (2011), which is incorporated by reference. For example, a look-up table may include the temperature detection value of 98.6 degrees F. as “negative” and the temperature detection value of 100 degrees F. as “positive.” For example, a look-up table may include a florescence value above a predetermined background level as “positive,” and a florescence value below a predetermined background level as “negative.” In some embodiments, the processor can include a look up table that incorporates values from both the pathogen detection region and the temperature detector that are identified as diagnostically “positive” or “negative” or “indeterminate.” For example, a combination of temperature detection value of 100 degrees F. and a florescence value above a predetermined background level may be classified as “positive.” For example, a combination of temperature detection value of 98.6 degrees F. and a florescence value below a predetermined background level may be classified as “negative.” For example, a combination of temperature detection value of 100 degrees F. and a florescence value below a predetermined background level may be classified as “indeterminate.” Some embodiments include a visual indicator attached to the processor, the visual indicator configured to be responsive to signals from the processor. For example, a visual indicator can include three LED lights of different colors, and the processor can be configured to send a signal to illuminate each color in combination with the results of the look up table. For example, a visual indicator can include LED lights colored blue, green and red. For example, a visual indicator can include a graphics display unit, such as an e-ink device.

The test units described herein provide persistent visual results of multiple diagnostic parameters on each test unit. Results from a test unit can be quickly evaluated by a healthcare provider as an initial screening tool for infection. “Persistent,” as used herein, refers to visual indicators on the test units that remain in position and visible to an observer for no less than 30 minutes. A persistent result on a test unit appears after the test unit is initially used with an individual patient, and remains in place on the test unit for no less than 30 minutes. For example, in some embodiments, a persistent result lasts for no less than 30 minutes. For example, in some embodiments, a persistent result lasts for no less than 45 minutes. For example, in some embodiments, a persistent result lasts for no less than 1 hour.

In some embodiments, a test unit includes a removable cover, the cover configured to inhibit the temperature detector and the at least one pathogen detection region during storage of the test unit. For example, the removable cover can include metalized plastic substantially enclosing the test unit. For example, the removable cover can include one or pieces of adhesive-backed paper covering the temperature detector and the at least one pathogen detection region on the solid support of the test unit. See, e.g. U.S. Pat. No. 5,401,100, “Auxillary Thermometer Packaging,” to Thackston and Focarino, which is incorporated by reference.

In some embodiments, the test unit is configured to be single-use. In some embodiments, the test unit is configured to be disposable. In some embodiments, the test unit is configured to be single-use and disposable. For example, a test unit can be fabricated from inexpensive and readily disposable materials, such as plastic and paper materials. For example, a test unit can be fabricated to include detection agents and visible indicators of the detection agents that are inexpensive and readily disposable. For example, a test unit can be fabricated to include detection agents and visible indicators of the detection agents including chemically-labeled MAbs and corresponding capture MAbs affixed to a nitrocellulose membrane. For example, a test unit can be fabricated to include a chemically-based temperature detector and corresponding temperature indicator. In some embodiments, a test unit is fabricated with substantially non-toxic materials. In some embodiments, a test unit is fabricated with substantially bio-compatible materials.

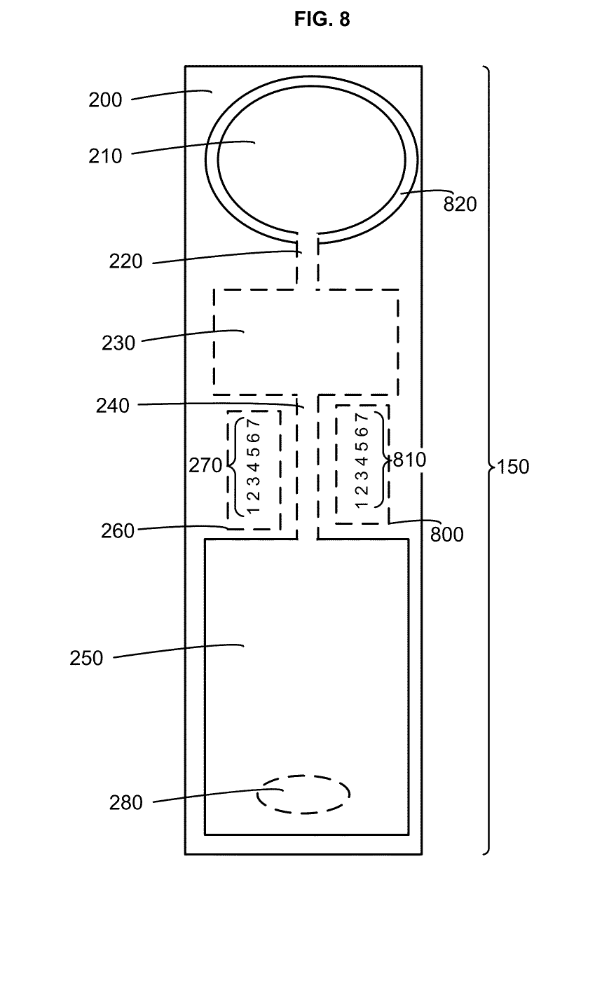

In some embodiments, a test unit includes a sensor of a physiological condition affixed to the solid support; and an indicator attached to the sensor of a physiological condition. See, e.g., FIG. 8. For example, in some embodiments a test unit includes a sensor of a physiological condition including a sensor of sweat level on the skin surface of the individual at a location adjacent to the solid support, and an attached electronic indicator. See, e.g. Katoh et al., “Thermal-Based Skin Moisture Device with Contact Pressure Sensor,” Proceedings of IEEE International Conference on Micro Electro Mechanical Systems—MEMS, 276-279 (2010), which is incorporated by reference.

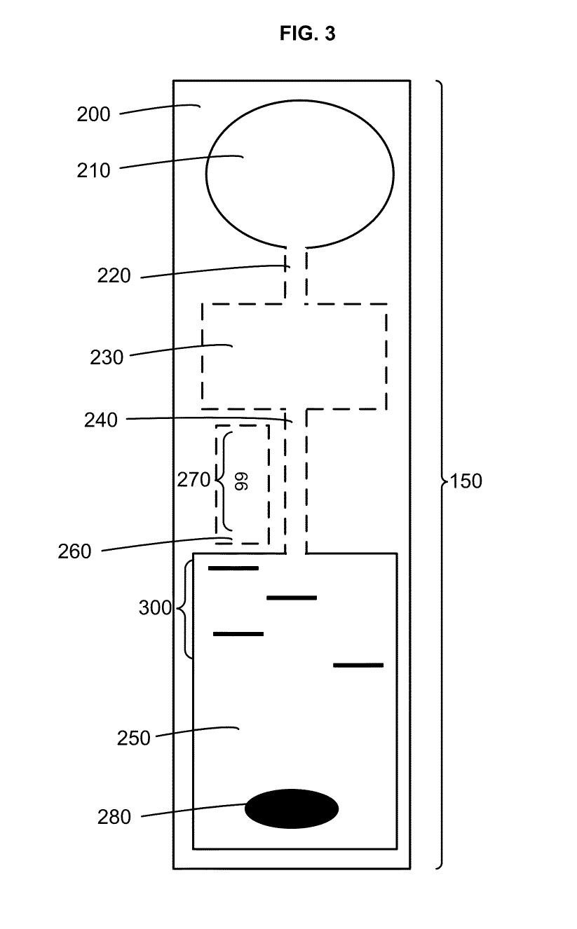

FIG. 3 illustrates aspects of an embodiment of a test unit 150. The test unit 150 shown in FIG. 3 has been used by a health care professional to provide analysis regarding a patient. The test unit 150 shown in FIG. 3 has received a body fluid, such as saliva, from a patient in the cavity 210 in the solid support 200. In the view illustrated in FIG. 3, the location on the surface of the solid support 200 adjacent to the internal reaction chamber 230, the first internal channel 220, and the second internal channel 240 is shown with a dotted line for illustration purposes. In some embodiments, these structures would normally not be visible, or not completely visible, through the outer surface of the solid support 200. In the illustration of the test unit 150 shown in FIG. 3, the body fluid has moved through the first internal channel 220 to the reaction chamber 230 internal to the solid support 200. The body fluid has mixed with a plurality of detection agents within a space internal to the reaction chamber 230. In the embodiment illustrated, the detection agents include a plurality of MAbs labeled with colloidal gold particles that specifically bind to influenza pathogen proteins, more specifically proteins specific to both strains A and B influenza, as well as proteins specific to either A or B strains of influenza. The mixture of body fluid and detection agents then moved through the second internal channel 240 to the one pathogen detection region 250 internal to the solid support 200. In the embodiment illustrated, a transparent plastic film covers the one pathogen detection region 250 internal to the solid support 200, so that color changes on the pathogen detection region 250 can be seen by an outside observer, such as a healthcare provider. The pathogen detection region 250 includes a plurality of visual indicators 300 indicating a positive reaction to a plurality of influenza-specific proteins. These visual indicators 300 change color when the MAbs labeled with colloidal gold particles and bound to influenza proteins bind to capture MAbs localized at the sites of the visual indicators 300. A healthcare provide could interpret the plurality of visual indicators 300 to assist with making a diagnosis of influenza. The pathogen detection region 250 also includes a positive control region 280 to verify assay results. The positive control region 280 can include, for example, a capture agent that includes a MAb that binds to a saliva-specific protein, wherein the capture agent has been immobilized on the pathogen detection region 250 at a specific location.

In the embodiment illustrated in FIG. 3, the test unit 150 includes a temperature detector 260 affixed to the solid support 200. The temperature detector 260 in the illustrated embodiment is an electronic temperature detector affixed to a location on the solid support adjacent to the surface configured to reversibly mate with the skin of an individual, i.e. the reverse side of the solid support as illustrated in FIG. 3. The temperature detector 260 is not visible in FIG. 3, however the corresponding location on the solid support is indicated with a dotted line. A wire (not shown) is positioned within the solid support to connect the temperature detector 260 with a persistent visible temperature indicator 270 on the opposing side of the solid support 200, i.e. the side of the solid support shown in FIG. 3. In the embodiment shown in FIG. 3, the persistent visible temperature indicator 270 is an electronic persistent visible temperature indicator. In some embodiments, a power source such as a battery is attached to an electronic temperature detector and/or an electronic persistent visible temperature indicator to form circuitry for detecting the body temperature of an individual and providing a persistent visible temperature indicator of the results of the detection.

FIG. 4 illustrates an embodiment of a test unit 150. In the view shown in FIG. 4, the test unit is depicted in a cross-section through the approximate center of the long axis of the test unit 150. The test unit 150 is fabricated as a series of substantially planar layers 420, 430, 440 affixed to each other on their largest surfaces. The test unit 150 includes a surface configured to be positioned adjacent to the surface of an individual's skin, which is on the right side in the view shown in FIG. 4. The test unit 150 includes a surface configured to be positioned distal to the surface of an individual's skin, which is on the left side in the view shown in FIG. 4. The surface configured to be positioned distal to the surface of an individual's skin (i.e. the left side as shown in FIG. 4) is also configured to be examined by a healthcare professional to determine the results of the test unit after use.

The test unit 150 shown in FIG. 4 includes a solid support 200 made up of a series of substantially planar layers 420, 430, 440. A first layer 420 includes a surface configured to be positioned distal to the surface of an individual's skin (i.e. the left side as shown in FIG. 4). The layer 420 is fabricated from a substantially translucent material, such as a thin plastic sheet or a thin paper sheet. The layer 420 includes an aperture which forms part of the cavity 210 in the solid support 200. An electronic persistent visible temperature indicator 270 is affixed to the surface configured to be positioned distal to the surface of an individual's skin (i.e. the left side as shown in FIG. 4). The electronic persistent visible temperature indicator 270 is connected to a first end of a wire connector 410. The second end of the wire connector 410 is connected to an electronic temperature detector 260 affixed to the solid support 200 on the surface configured to be positioned adjacent to the surface of an individual's skin, which is on the right side in the view shown in FIG. 4.

A second layer 430 of the solid support 200 is positioned between the first layer 420 and a third layer 440, with surfaces of the second layer 430 affixed to respective surfaces of the first layer 420 and the third layer 440. The second layer 430 includes an aperture which forms part of the cavity 210 in the solid support 200, the aperture in the second layer 430 positioned adjacent to the aperture in the first layer 420 to form the cavity 210 in the solid support 200. The second layer 430 includes a first internal channel 220 in the solid support 200, the first internal channel 220 connected at a first end to the cavity 210, the first internal channel 220 connected at a second end to the reaction chamber 230. In some embodiments, the first internal channel 220 includes fluid-control film oriented to permit directional flow of fluid from the cavity 210 to the reaction chamber 230. See U.S. Pat. No. 6,420,622 to Johnston et al., “Medical Article Having Fluid Control Film,” which is incorporated by reference.

The reaction chamber 230 is formed from a space in the second layer 430 of the solid support, the space in the second layer 430 forming a gap between the first layer 420 and the third layer 440 of the solid support 200. In some embodiments, the reaction chamber 230 includes an enclosure between the first layer 420 and the third layer 440 of the solid support 200, such as an enclosure fabricated from a thin plastic material. The reaction chamber 230 includes a plurality of detection agents within the space between the first layer 420 and the third layer 440 of the solid support 200. For example, in some embodiments the reaction chamber 230 includes plurality of detection agents that include MAbs labeled with colloidal gold particles, the MAbs specific to proteins from a pathogen, such as influenza. For example, in some embodiments the reaction chamber 230 includes plurality of detection agents that include MAbs labeled with colloidal gold particles, the MAbs specific to proteins within the body fluid of interest, such as inflammation response proteins. For example, in some embodiments the reaction chamber 230 includes plurality of detection agents that include MAbs labeled with colloidal gold particles, the MAbs specific to proteins within the body fluid of interest, such as structural proteins (e.g. to serve as one or more positive controls).

The second layer 430 of the solid support 200 includes a second internal channel 240 in the solid support 200, the second internal channel 240 connected at a first end to the reaction chamber 230, the second internal channel 240 connected at a second end to a pathogen detection region 250. In some embodiments, the second internal channel 240 includes fluid-control film oriented to permit directional flow of fluid from the reaction chamber 230 to the at least one pathogen detection region 250. See U.S. Pat. No. 6,420,622 to Johnston et al., “Medical Article Having Fluid Control Film,” which is incorporated by reference.

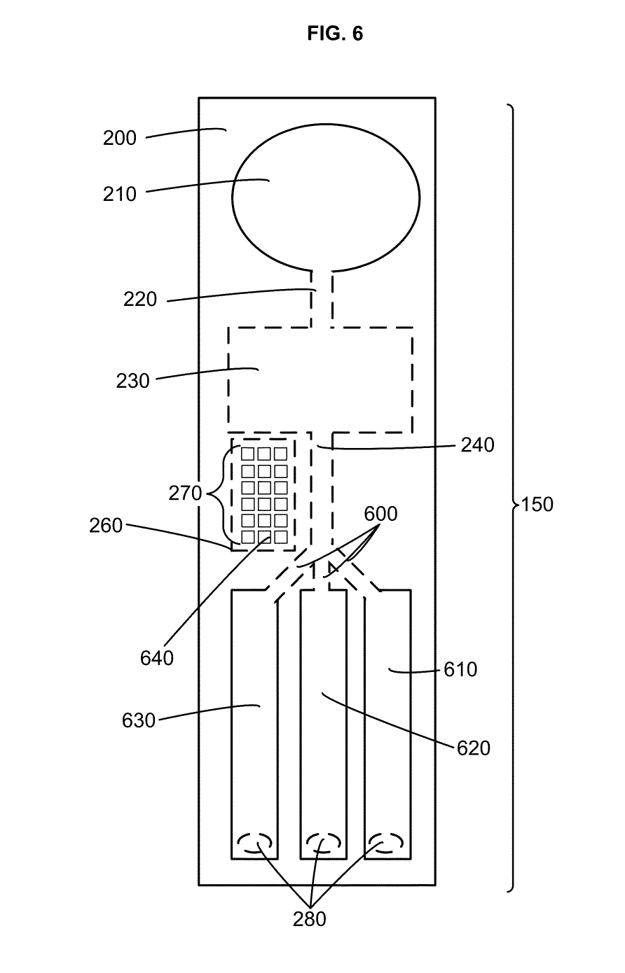

The second layer 430 of the solid support 200 includes a pathogen detection region 250. In some embodiments, a test unit includes a single pathogen detection region. In some embodiments, a test unit includes a plurality of pathogen detection regions (see, e.g. FIG. 6). The pathogen detection region 250 within the second layer 430 of the solid support 200 includes a space in the second layer 430 of the solid support 200, forming a gap between the first layer 420 and the third layer 440 of the solid support 200. In some embodiments, there is a structural wall surrounding at least a portion of the pathogen detection region. For example, some embodiments include a thin plastic sheet positioned adjacent to the surface of the first layer and the third layer of the solid support, the thin plastic sheet substantially enclosing the pathogen detection region. The pathogen detection region 250 includes at least one visible indicator of one or more of the plurality of detection agents found in the reaction chamber 230. Some embodiments include visible indicators including a plurality of MAbs affixed to specific locations on a nitrocellulose membrane, the MAbs affixed to the membrane specific for proteins corresponding to those of labeled MAbs in the reaction chamber.

In the embodiment illustrated in FIG. 4, the test unit 150 solid support 200 includes a third layer 440. The third layer 440 includes a surface configured to reversibly mate with a skin surface of an individual (e.g. the right side as shown in FIG. 4). Some embodiments include a bio-compatible adhesive on the surface configured to reversibly mate with a skin surface of an individual. The third layer 440 includes a temperature detector 260 integrated within the third layer 440. In the embodiment shown in FIG. 4, the temperature detector 260 is an electronic temperature detector which is attached to an electronic temperature indicator 270 with a wire connector 410. Although the wire connector 410 is illustrated as two parts in FIG. 4, the illustration should be construed as including the wire 410 bending around the second internal channel 240, and therefore being partially out of the view shown in FIG. 4. The temperature detector 260 integrated within the third layer 440 includes a surface configured to reversibly mate with a skin surface of an individual, the surface of the temperature detector 260 being included within the surface configured to reversibly mate with a skin surface of an individual of the third layer 440 of the solid support 200.

FIG. 5 illustrates aspects of an embodiment of a test unit 150 after use to analyze a body fluid sample from an individual patient. In the view illustrated in FIG. 5, the test unit is depicted in a cross-section through the approximate center of the long axis of the test unit 150. The test unit 150 is fabricated as a series of substantially planar layers 420, 430, 440 affixed to each other on their largest surfaces. The test unit 150 includes a surface configured to be positioned adjacent to the surface of an individual's skin, which is on the right side in the view shown in FIG. 5. The test unit 150 includes a surface configured to be positioned distal to the surface of an individual's skin, which is on the left side in the view shown in FIG. 5. The surface configured to be positioned distal to the surface of an individual's skin (i.e. the left side as shown in FIG. 5) is also configured to be examined by a healthcare professional to determine the results of the test unit after use. For example, the first layer 420 of the test unit 150 includes a transparent material 500 covering the surface configured to be positioned distal to the surface of an individual's skin over the pathogen detection region 250 of the test unit 150. For example, some embodiments include a transparent material such as a thin film of transparent plastic covering the pathogen detection region, the edges of the thin film affixed to the support structure around the pathogen detection region.

The test unit 150 shown in FIG. 5 has been used with a sample of body fluid, such as saliva or nasal fluid, from an individual patient. The dotted arrows in FIG. 5 illustrate the movement of the body fluid through the test unit during use. The body fluid entered the test unit through the cavity 210 in the solid support 200. The cavity 210 is of a size and shape to hold a sufficient volume of body fluid for analysis with the test unit 150, e.g. a sufficient volume to travel through the first internal channel 220, mix with the detection agents in the reaction chamber 230, move through the second internal channel 240, and activate the visible indicator of one or more of the plurality of detection agents in the pathogen detection region 250 internal to the solid support 200. After entering the test unit 150 through the cavity 210, the body fluid passed through the first internal channel 220 to the reaction chamber 230. The reaction chamber 230 included a plurality of detection agents within a space before contact with the body fluid. After contact, the body fluid mixed with the plurality of detection agents. The body fluid mixture then moved through the second internal channel 240 into the pathogen detection region 250.

Within the pathogen detection region 250 the body fluid mixture activated a first visible indicator 520, a second visible indicator 530 and a third visible indicator 540. The visible indicators 520, 530, 540 are positioned at locations on the pathogen detection region in a pattern, so that a healthcare provider can recognize the pattern and identify which specific proteins were detected in the body fluid sample. For example, in some embodiments a first visible indicator 520 can correspond to an influenza protein common to many strains of influenza, a second visible indicator 530 can correspond with a protein specific to influenza A, and the third visible indicator 540 can correspond to an inflammatory response protein in humans. Having all three of these visible indicators active or visible in a test unit, as illustrated in FIG. 5, would indicate to a healthcare professional that the patient providing the body sample shows signs of an influenza infection. The pathogen detection region of a test unit can include visible indicators that are not active after contact with a body fluid mixture, such as those that would become visible if pathogen proteins are present, but the pathogen proteins are not present in a specific body fluid sample. For example, in some embodiments a first visible indicator can correspond to an influenza protein common to many strains of influenza, a second visible indicator can correspond with a protein specific to influenza A, and the third visible indicator can correspond to an inflammatory response protein in humans. If none of the first, second and third visible indicators are active, a healthcare professional examining the test unit after use may understand that the patient who provided the sample does not show signs of infection from influenza. The test unit 150 shown in FIG. 5 also includes a visual indicator of a positive control protein 280. In the embodiment illustrated in FIG. 5, the visual indicator of a positive control protein 280 is active, making a visible sign to a healthcare professional that the test unit was operational when used.

The embodiment of a test unit 150 shown in FIG. 5 includes a temperature detector 260 affixed to the solid support 200 at a position adjacent to the surface configured to reversibly mate with a skin surface of an individual (e.g. to the left as illustrated in FIG. 5). In the embodiment illustrated, the temperature detector 260 is an electronic temperature detector. The electronic temperature detector is connected to a persistent visible temperature indicator 270 with a wire connector 410. The persistent visible temperature indicator 270 shown in FIG. 5 is an electronic persistent visible temperature indicator. Although in the view shown in FIG. 5 it appears that the wire connector 410 is in two sections, the wire connector 410 is a single connector that passes around the second internal channel 240 and, therefore, is partially obscured from the view illustrated. A power source, such as a battery, can be operably connected to the electronic temperature detector and the electronic persistent visible temperature indicator. The persistent visible temperature indicator 270 is affixed to the surface of the first layer 420 of the solid support 200 at a position adjacent to the transparent cover 500 over the pathogen detection region 250. This positioning places the visible indicators of the test unit results near each other for convenient evaluation and documentation by a healthcare professional. In some embodiments, the used test unit will be photographed or scanned for storage in an individual patient's electronic medical record.

FIG. 6 illustrates aspects of an embodiment of a test unit 150. The test unit 150 shown in FIG. 6 is configured for use with nasal fluid from an individual. The cavity 210, for example, is of a size and shape to accept and retain a quantity of nasal fluid from an individual. Some embodiments of a test unit include, for example, a nasal fluid receiving cavity including an indentation in a surface of the solid support, the indentation configured to retain nasal fluid directly from a nasal cavity of an individual. For example, the cavity may include a flange or edge for the individual user to position their nose along when dispensing a nasal fluid sample to the cavity. For example, the cavity may have an oval shape configured to approximate the size and shape of an individual's nasal opening. Some embodiments include a nasal fluid receiving cavity in the solid support including an indentation in a surface of the solid support, the indentation configured to retain no more than approximately 1 milliliter of nasal fluid. Some embodiments include a nasal fluid receiving cavity in the solid support including an indentation in a surface of the solid support, the indentation configured to retain no more than approximately 100 microliters of nasal fluid.

As shown in FIG. 6, the test unit 150 configured for use with a nasal fluid includes a solid support 200 including a surface configured to reversibly mate with the skin of an individual. The surface configured to reversibly mate with the skin of an individual is located on the face of the solid support 200 opposing the face visible in FIG. 6. In the illustrated embodiment, the solid support 200 is a substantially flat solid support. The test unit 150 includes a nasal fluid receiving cavity 210 in the solid support 200. For example, in some embodiments, the nasal fluid receiving cavity includes an edge region of a size and shape to approximate the nasal opening in an individual's nose. For example, in some embodiments, the nasal fluid receiving cavity includes an edge region of a size and shape to assist an individual to position his or her nose adjacent the nasal fluid receiving cavity.

The test unit 150 shown in FIG. 6 includes components that are interior to the solid support 200. These components are not always visible from an external view, for example in embodiments wherein the solid support is fabricated from a non-transparent material, such as a non-transparent plastic or paper material. In some embodiments, the components interior to the solid support 200 are visible, for example in embodiments wherein the solid support is partially or completely fabricated from a transparent plastic or paper material. In the embodiment illustrated in FIG. 6, the solid support 200 is fabricated with a support layer including a surface configured to reversibly mate with the skin of an individual and an opposing surface with channels and indentations positioned to form the walls of the other components of the test unit 150. The solid support 200 also includes a transparent layer covering the surface of the support layer opposing the surface configured to reversibly mate with the skin of an individual. The transparent layer and the support layer of the solid support are affixed to each other at their adjacent faces. An individual observer, such as a healthcare provider, can therefore see the interior components of the test unit through the transparent layer. In FIG. 6, the interior components covered with the transparent layer are shown with dotted lines for purposes of illustration. In some embodiments, for example, a support layer of a solid support is fabricated from polypropylene. In some embodiments, for example, a transparent layer of a solid support is fabricated from a thin transparent plastic material.

The test unit 150 illustrated in FIG. 6 includes a reaction chamber 230 internal to the solid support 200. The reaction chamber includes a plurality of detection agents within a space within the solid support. The detection agents included in a reaction chamber of a test unit configured for use with nasal fluid include detection agents for pathogens present in nasal fluid. For example, the detection agents included in a reaction chamber of a test unit configured for use with nasal fluid can include those that react with influenza proteins. For example, in some embodiments the detection agents included in a reaction chamber of a test unit configured for use with nasal fluid can include MAbs with affixed colloidal gold particles, the MAbs specific for binding to proteins from multiple influenza strains, a class of influenza strains (e.g. influenza A or influenza B), or specific influenza strains (e.g. H1N1).

In the embodiment shown in FIG. 6, the test unit 150 includes a plurality of pathogen detection regions 610, 620, 630 internal to the solid support 200. The test unit 150 illustrated in FIG. 6 includes a first pathogen detection region 610, a second pathogen detection region 620 and a third pathogen detection region 630. Each of the 3 pathogen detection regions 610, 620, 630 include a visible indicator of one or more of the plurality of detection agents from the reaction chamber 230. Some embodiments include at least one pathogen detection region internal to the solid support, the pathogen detection region including: a membrane; and a plurality of capture agents affixed to the membrane in a pattern. For example, in some embodiments each pathogen detection region includes a membrane with one or more groups of MAbs affixed to the membrane in specific locations. Each of the groups of MAbs are configured to specifically bind to a pathogen protein that is also the target of labeled MAbs in the detection agents within the associated reaction chamber. For example, in some embodiments the detection agents within the reaction chamber of a test unit include MAbs labeled with colloidal gold particles directed to influenza A specific protein, and a pathogen detection region of the same test unit includes a visible indicator including MAbs affixed to a membrane, the MAbs of the visible indicator including those specific for the same influenza A specific protein as the labeled MAbs of the detection agents within the associated reaction chamber. Some embodiments include one or more pathogen detection regions including at least one control visual indicator 280. In the embodiment illustrated in FIG. 6, each of the 3 pathogen detection regions 610, 620, 630 include a control visual indicator 280. For example, a control visual indicator can include a pH-sensitive paper, configured to change color when contacted with a fluid of the approximate pH of nasal fluid. For example, a control visual indicator can include a paper that changes color when wet, which when positioned at the end of the pathogen control region distal to the second internal channel indicates to a user that the fluid has permeated the length of the pathogen detection region.

The test unit 150 shown in FIG. 6 also includes a first internal channel 220 to the solid support 200, the first internal channel 220 including a first end attached to the nasal fluid receiving cavity 210, the first internal channel 220 including a second end attached to the reaction chamber 230. In the embodiment illustrated, the test unit 150 also includes a second internal channel 240 to the solid support 200, the second internal channel 240 including a first end attached to the at least one reaction chamber 230, the internal channel including 3 second ends, each of the second ends attached to one of the three pathogen detection regions 610, 620, 630 internal to the solid support 200. The second internal channel 240 included in the embodiment illustrated in FIG. 6 includes a plurality of ends, each end connected to a separate pathogen detection region.

In the embodiment shown in FIG. 6, the temperature detector 260 affixed to the solid support 200 includes a region of the solid support 200 that provides thermal transfer from the surface configured to reversibly mate with the skin of an individual. The embodiment shown includes a chemical-based temperature detector 260 attached to a chemical-based persistent visual temperature indicator 270. The chemical-based temperature detector 260 includes a plurality of indicator wells (e.g. item labeled as 640). Each of the indicator wells can be filled with a chemical substance that changes color in response to a specific temperature. The combination of a plurality of indicator wells filled with a temperature-reactive chemical substance can indicate to a user the temperature range of a patient's skin surface. See e.g., U.S. Pat. Nos. 4,232,552, 4,339,207 and 4,362,645, each titled “Temperature Indicating Compositions of Matter,” to Hof et al., which are each incorporated herein by reference. See U.S. Pat. No. 5,816,707, “Reversible Chemical Thermometer,” to Hof, which is incorporated by reference. In some embodiments, a temperature detector affixed to a solid support of a test unit configured for use with a nasal fluid includes an electronic temperature detector, and an electronic persistent visible temperature indicator attached to the electronic temperature detector.

In some embodiments, a test unit configured for use with a nasal sample includes a removable cover, the cover configured to inhibit the temperature detector and the at least one pathogen detection region during storage of the test unit. For example, the removable cover can include thermal insulation material positioned to inhibit temperature variations in the temperature detector and the at least one pathogen detection region during storage of the test unit. For example, the removable cover can include metalized plastic material. In some embodiments, a test unit configured for use with a nasal sample is configured to be single-use. For example, the detection agents and/or the visual indictors of the detection agents can be included in a concentration that will be consumed by a single use of the test unit.

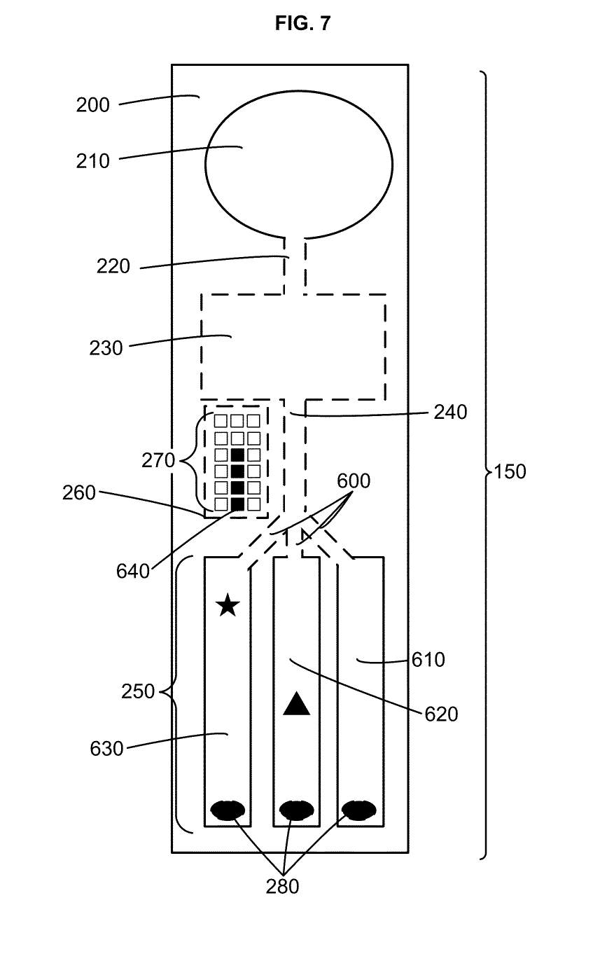

FIG. 7 illustrates aspects of a test unit 150 configured for analysis of a nasal fluid sample after use, as it would be examined by a user, such as a healthcare provider. In the embodiment shown, the second pathogen detection region 620 and the third pathogen detection region 630 both include visual indicators of the detection of a pathogen protein present in the nasal fluid of the individual (e.g. the triangle and star mark, respectively). Each of the first pathogen detection region 610, the second pathogen detection region 620 and the third pathogen detection region 630 include a control visual indicator 280. The color change in each control visual indicator 280 shows that the associated pathogen detection region, and therefore the test unit, have undergone a reaction and are not available for re-use.

FIG. 7 also illustrates a temperature detector 260 affixed to the solid support 200, the temperature detector including a plurality of wells (e.g. 640) in the solid support, the wells in thermal contact with the surface of the solid support configured to reversibly mate with the skin of an individual. Each of the wells includes a persistent visible temperature indicator that is a chemical substance that changes color at a specific temperature. See e.g., U.S. Pat. Nos. 4,232,552, 4,339,207 and 4,362,645, each titled “Temperature Indicating Compositions of Matter,” to Hof et al., which are each incorporated herein by reference. See U.S. Pat. No. 5,816,707, “Reversible Chemical Thermometer,” to Hof, which is incorporated by reference. See e.g., U.S. Pat. Nos. 7,875,207 and 8,083,969, each titled “Thermally-Responsive Materials and Devices Comprising Such Materials,” to Stewart, which are each incorporated herein by reference. In the embodiment shown, a series of wells (e.g. 640) have changed color, which indicate to a healthcare provider the detected temperature range of the individual's skin.

FIG. 8 illustrates an embodiment of a test unit 150 configured for analysis of nasal fluid from an individual. The test unit 150 includes a solid support 200. The solid support 200 is fabricated from two layers of translucent polypropylene, the layers affixed to each other on their opposing faces. The test unit 150 includes internal structures (e.g. the first internal channel 220, the second internal channel 240, and the reaction chamber 230) which are partially visible through the translucent polypropylene layers, and are indicated as dotted lines in FIG. 8. The solid support includes a cavity 210 configured to receive and retain nasal fluid from an individual. The cavity 210 includes an edge region including a flange 820 aligned to receive and retain nasal fluid in a location adjacent to the cavity 210. The flange 820 is fabricated from a thin sheet of polypropylene curved around the edge of the cavity 210 and affixed to the edge around the circumference of the cavity 210.

A first internal channel 220 is positioned within the layers of the solid support 200. The first internal channel 220 includes a first end attached to the nasal fluid receiving cavity 210, and a second end attached to the reaction chamber 230. The first internal channel 220 is positioned and shaped to direct nasal fluid from the interior of the cavity 210 to the interior of the reaction chamber 230. A second internal channel 240 is positioned within the solid support, the second internal channel including a first end attached to the reaction chamber 230, the second internal channel including a second end attached to the pathogen detection region 250.

A reaction chamber 230 is positioned within the layers of the solid support 200. The reaction chamber 230 includes a plurality of detection agents within a space. In some embodiments, a reaction chamber includes a plurality of detection agents configured to detect a specific pathogen present in nasal fluid. For example, a reaction chamber can include a plurality of detection agents configured to detect influenza-specific proteins. For example, a reaction chamber can include a plurality of detection agents configured to detect rhinovirus specific proteins. The reaction chamber includes the plurality of detection agents positioned to mix with the nasal fluid when the nasal fluid flows through the first internal channel into the reaction chamber.

A pathogen detection region 250 is attached to the second end of the second internal channel 240. The at least one pathogen detection region includes at least one visible indicator of one or more of the plurality of detection agents. In the embodiment illustrated, the test unit has not been used, so the visible indicator is not visible. The pathogen detection region also includes a positive control indicator 280. The positive control indicator 280 is not visible in the embodiment illustrated since the test unit has not been used.

The embodiment of a test unit 150 shown in FIG. 8 also includes a temperature detector 260 attached to the solid support 200. The temperature detector is an electronic temperature detector affixed between the layers of the solid support, the electronic temperature detector oriented to detect temperature at a position adjacent to the surface of the solid support configured to reversibly mate with the skin of an individual. The temperature detector 260 is attached to a persistent visible temperature indicator 270. The persistent visible temperature indicator can include, for example, an electronic persistent visible temperature indicator. A persistent visible temperature indicator can include a thin-film e-ink based device.

In the embodiment shown in FIG. 8, the test unit 150 configured for analysis of a nasal fluid includes a sensor of a physiological condition 800 affixed to the solid support, and a visible indicator 810 attached to the sensor of a physiological condition. For example, in some embodiments a test unit includes a sensor of a physiological condition including a sensor of sweat level on the skin surface of the individual at a location adjacent to the solid support, and an attached electronic indicator. See, e.g. Katoh et al., “Thermal-Based Skin Moisture Device with Contact Pressure Sensor,” Proceedings of IEEE International Conference on Micro Electro Mechanical Systems—MEMS, 276-279 (2010), which is incorporated by reference.

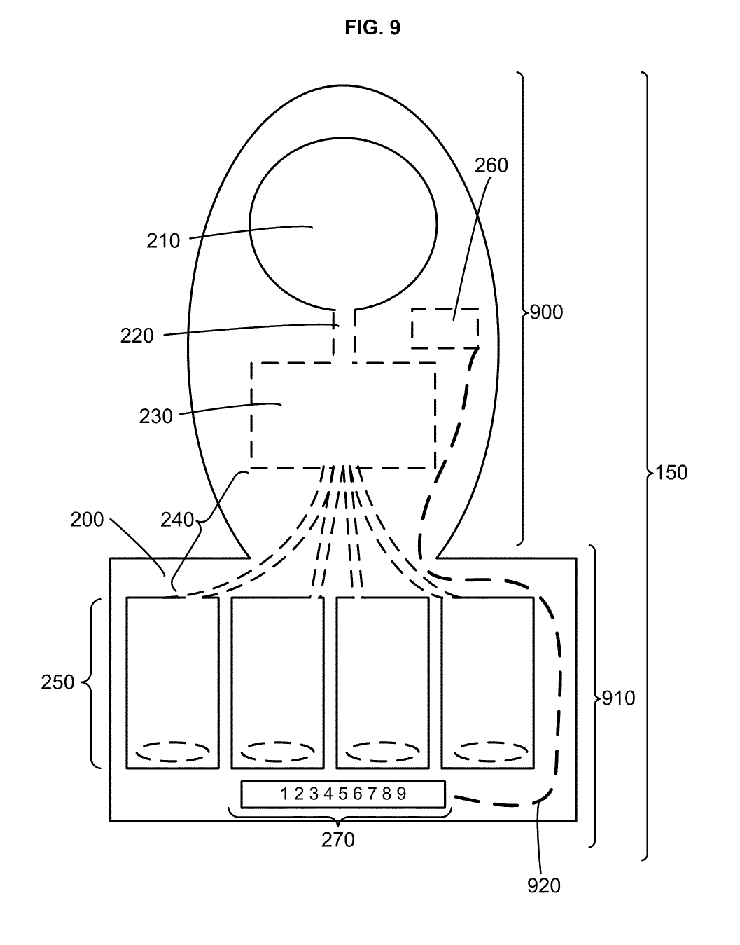

FIG. 9 illustrates an embodiment of a test unit 150 configured for detection of analytes in oral fluid (e.g. saliva). The test unit 150 includes a solid support 200 including a first region 900 configured to be enclosed within an oral cavity of an individual. The first region 900 configured to be enclosed within an oral cavity of an individual is configured in a size and shape to be positioned within the mouth of an individual. For example, the first region 900 of the solid support 200 configured to be enclosed within an oral cavity of an individual can be a substantially smooth solid support. In some embodiments, an embodiment of a test unit 150 configured for detection of analytes in oral fluid is fabricated from plastic, which retains its shape and size even after exposure to fluid (e.g. does not expand in the presence of fluid).

The test unit 150 includes an oral fluid receiving cavity 210 in the solid support 200. The oral fluid receiving cavity 210 is configured to receive and retain oral fluid when placed in an individual's mouth. In some embodiments, an oral fluid receiving cavity 210 includes an indentation in a surface of the solid support, the indentation configured to retain oral fluid directly from an oral cavity. In some embodiments, an oral fluid receiving cavity 210 includes an indentation in a surface of the solid support, the indentation configured to retain no more than approximately 1 milliliter of oral fluid. In some embodiments, an oral fluid receiving cavity 210 includes an indentation in a surface of the solid support, the indentation configured to retain no more than approximately 100 microliters of oral fluid. The test unit 150 also includes a second region 910 configured to be retained externally from the individual's mouth. For example, during use of the test unit 150, the first region 900 configured to be enclosed within an oral cavity of an individual can be placed in the individual's mouth, while the second region 910 remains external to the mouth. The persistent visible temperature indicator and the pathogen detection regions are positioned on the second region 910, allowing for an observer, such as healthcare provider, to see that the test unit 150 includes visible indicators even when it is still positioned in an individual's oral cavity.

The embodiment illustrated in FIG. 9, the oral fluid receiving cavity 210 in the solid support 200 is connected to a reaction chamber 230 internal to the solid support 200. Although the reaction chamber 230 is internal to the solid support 200, its approximate position is illustrated with dotted lines. The reaction chamber includes a plurality of detection agents within a space. The detection agents are configured to detect agents indicative of infection in a sample of oral fluid. Test systems for detection of oral analytes are described. See e.g., U.S. Pat. Nos. 7,700,305 and 8,067,188, each titled “Analyte Detection”, to Toranto, which are each incorporated herein by reference. See also U.S. Patent Application Publication Nos. 2010/0330684 and 2011/0287409, each titled “Diagnostic Device and Method,” to O'Connor, and U.S. Patent Application Publication No. 2012/0149124, “Device for Collection and Assay of Oral Fluids,” to Mink et al., which are each incorporated by reference. For example, in some embodiments, the reaction chamber includes MAbs affixed to colloidal gold particles that recognize inflammatory markers and microbial pathogens. For example, detection of C-reactive protein (CRP), interleukin 1-β, and β-glucuronidase in saliva can indicate an ongoing microbial infection derived from viral and bacterial sources. See e.g.: Miller et al., “Current Developments in Salivary Diagnostics”, Biomark Med. 4:171-189, (2010); Watson et al., “Raised Inflammatory Markers”, BMJ 344:e454, (2012), (doi: 10.1136/bmj.e454); and Mogensen, “Pathogen Recognition and Inflammatory Signaling in Innate Immune Defenses,” Clin. Microbiol. Rev. 22: 240-273, (2009) which are each incorporated herein by reference.

The oral fluid receiving cavity 210 in the solid support 200 illustrated in FIG. 9 is connected to the reaction chamber 230 through a first internal channel 220 in the solid support 200. Although the first internal channel 220 is internal to the solid support 200, its approximate position is illustrated with dotted lines. In some embodiments, a first internal channel includes a fluid-control film oriented to permit directional flow of fluid from the oral fluid receiving cavity 210 to the reaction chamber 230. See U.S. Pat. No. 6,420,622 to Johnston et al., “Medical Article Having Fluid Control Film,” which is incorporated by reference.

The reaction chamber 230 is connected to a series of pathogen detection regions 250 with a series of second internal channels 240. As shown in FIG. 9, a second internal channel 240 connects each of the pathogen detection regions 250 with the reaction chamber 230. Although series of second internal channels 240 are internal to the solid support 200, the approximate position is illustrated with dotted lines. In some embodiments, a second internal channel includes a fluid-control film oriented to permit directional flow of fluid from the reaction chamber 230 to the at least one pathogen detection region 250. See U.S. Pat. No. 6,420,622 to Johnston et al., “Medical Article Having Fluid Control Film,” which is incorporated by reference.

Each of the pathogen detection regions 250 includes a pathogen detector and at least one visible indicator of one or more of the plurality of detection agents in the reaction chamber 230. For example, in some embodiments each of the pathogen detection regions 250 includes a visible indicator including nitrocellulose membrane with affixed MAbs in an array, the MAbs known to bind to specific pathogen proteins and inflammatory proteins known to be present in oral fluid. The reaction chamber can include a plurality of detection agents including MAbs with affixed colloidal gold particles, the MAbs known to bind to the same specific pathogen proteins and inflammatory proteins as the MAbs affixed within each of the pathogen detection regions. In order to form a positive, visible result of the assay, detection agents including MAbs with affixed colloidal gold particles would bind to a specific protein indicative of a pathogen or inflammatory response within the reaction chamber. The mixture of oral fluid and detection agents would then move to the pathogen detection region, where the MAbs affixed to the membrane would bind and capture the protein-labeled MAb combination at that location on the membrane to form a visible indicator. In the embodiment illustrated in FIG. 9, each of the plurality of pathogen detection regions 250 includes a transparent cover over the visible indicator of one or more of the plurality of detection agents, allowing a user, such as a healthcare provider, to see the visible indicator.

Some embodiments, such as the one illustrated in FIG. 9, include a plurality of pathogen detection regions 250 attached to a single reaction chamber 230 through a series of second internal channels 240. In some embodiments, each of the plurality of pathogen detection regions 250 includes the same visible indicators of the same subset of the plurality of detection agents, so that each of the plurality of pathogen detection regions 250 detects and provides visible indicators for the presence of the same proteins in the sample. Embodiments including a plurality of pathogen detection regions with substantially identical visible indicators of the same subset of the plurality of detection agents may be desirable in embodiments where confirmation of the result is required through a plurality of visible indicators of detection.

In some embodiments, each of the plurality of pathogen detection regions 250 includes visible indicators of a different subset of the plurality of detection agents, so that each of the plurality of pathogen detection regions 250 detects and provides visible indicators for the presence of different proteins in the sample. Embodiments including a plurality of pathogen detection regions with different visible indicators of different subsets of the plurality of detection agents may be desirable, for example, to provide a clear distinction between the visible indicators for a user. For example, each single pathogen detection region in a series of a plurality of pathogen detection regions can include one or more visible indicators of a specific pathogen or of inflammation. For example, a first pathogen detection region can include visible indicators of the presence of influenza proteins, while a second single pathogen detection region can include visible indicators of the presence of rhinovirus proteins, a third pathogen detection region can include visible indicators of the presence of inflammation. For example, detection of C-reactive protein (CRP), interleukin 1-β, and β-glucuronidase in saliva can indicate an ongoing microbial infection derived from viral and bacterial sources. See e.g.: Miller et al., “Current Developments in Salivary Diagnostics”, Biomark Med. 4:171-189, (2010); Watson et al., “Raised Inflammatory Markers”, BMJ 344:e454, (2012), (doi: 10.1136/bmj.e454); and Mogensen, “Pathogen Recognition and Inflammatory Signaling in Innate Immune Defenses,” Clin. Microbiol. Rev. 22: 240-273, (2009) which are each incorporated herein by reference. The separation of visible indicators of different pathogens as well as inflammation can provide a convenient way for a user, such as a healthcare provider, to interpret the visual indicator(s). For example, it may be easy for a user to see that there are visible indicators present in the second pathogen detection region of a used test unit, corresponding to a positive result for rhinovirus. In addition, it may be easy for a user to see that there are no visible indicators present in the first pathogen detection region of a used test unit, corresponding to a negative result for influenza.

As illustrated in FIG. 9, a test unit 150 configured for oral use includes a temperature detector 260 affixed to the solid support 200. The temperature detector 260 is affixed to the solid support 200 in the first region 900 configured to be enclosed within an oral cavity of an individual, in order to detect a temperature reading from within the oral cavity. The temperature detector 260 illustrated in the embodiment of FIG. 9 is an electronic temperature detector. The temperature detector 260 is connected to a persistent visible temperature indicator 270 with a wire connector 920. The persistent visible temperature indicator 270 shown in FIG. 9 is an electronic persistent visible temperature indicator.

FIG. 10 illustrates an embodiment of a test unit 150 with a solid support 200 including a region 900 configured to be enclosed within an oral cavity of an individual. The region 900 configured to be enclosed within an oral cavity of an individual includes an oral fluid receiving cavity 210 in the solid support 200. In the view shown in FIG. 10, the test unit 150 has been used, i.e. it has been placed in the oral cavity of an individual and oral fluid has entered the cavity 210, flowed through the first internal channel 220 in the solid support 200, mixed with a plurality of detection agents within the space of the reaction chamber 230, the mixture divided and moved through the second internal channels into the plurality of pathogen detection regions 250 internal to the solid support 200. Two of the plurality of pathogen detection regions 250 shown in FIG. 10 include a visible indicator of one or more of the plurality of detection agents. A pathogen detection region includes a first visible indicator 1000, while another pathogen detection region includes a second visible indicator 1010. All of the pathogen detection regions 250 include a positive control indicator 280, showing a user that oral fluid mixed with detection agents from the reaction chamber have flowed to those positions in the pathogen detection regions 250.

FIG. 10 includes a temperature detector 260 affixed to the solid support 200 in the region 900 configured to be enclosed within an oral cavity of an individual. The temperature detector 260 is attached to a persistent visible temperature indicator 270 with a wire connector 920. The persistent visible temperature indicator 270 shown in FIG. 10 presents a reading from the temperature detector.

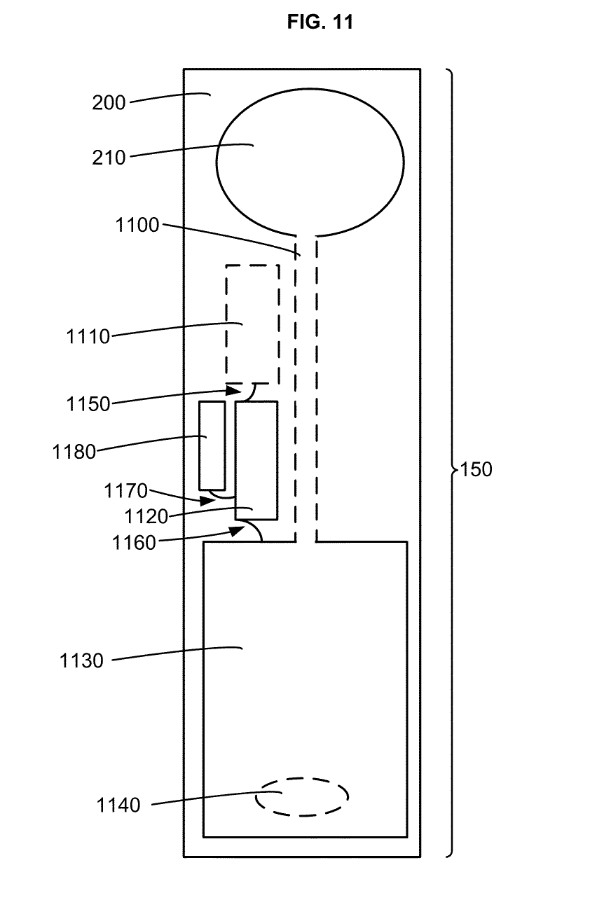

FIG. 11 illustrates an embodiment of a test unit 150. The test unit 150 includes a solid support 200 including a surface configured to reversibly mate with a skin surface of an individual. In the view shown, the surface configured to reversibly mate with a skin surface of an individual is on the reverse side from the side shown. The test unit 150 includes a cavity 210 in the solid support 200. In some embodiments, the cavity 210 includes a flange configured to enclose a nasal fluid. In some embodiments, the cavity 210 includes a flange configured to enclose an oral fluid.

The test unit 150 includes a pathogen detection region 1130 internal to the solid support 200. The pathogen detection region includes an electronic detector of at least one analyte. In some embodiments, the analyte includes analytes indicative of the presence of pathogens. For example, an analyte can include an influenza-specific protein. In some embodiments, the analyte includes analytes indicative of the presence of an inflammatory response in the individual. For example, in embodiments configured for use with saliva, an analyte can include C-reactive protein (CRP), interleukin 1-β, and β-glucuronidase. In some embodiments, the electronic detector detects an analyte directly. For example, in some embodiments, an electronic detector includes at least one wireless complementary metal oxide semiconductor (CMOS) sensor. See, for example, US Patent Application Publication No. 2009/0298704 to Anwar, “Wireless CMOS Biosensor,” which is incorporated herein by reference. See also: Daniel et al., “Implantable Diagnostic Device for Cancer Monitoring,” Biosens. Bioelectron. 24:11, 3252-3257 (2009); and Ling et al., “Implantable Magnetic Relaxation Sensors Measure Cumulative Exposure to Cardiac Biomarkers,” Nature Biotechnology 29: 3 273-278, which are each incorporated herein by reference. For example, in some embodiments an electronic detector includes an optically readable polydeoxy-nucleotide array with integral fluorescence excitation and fluorescence emission channels. See, for example, U.S. Pat. No. 7,302,289 to Crowley, “Readable Probe Array for In-Vivo Use,” which is incorporated herein by reference. In some embodiments, the electronic detector detects an analyte indirectly, such as through binding of a detectable agent such as a labeled antibody. A electronic detector can include a recognition element including at least one aptamer configured to bind to an analyte. In some embodiments, the electronic detector includes aptamer-based detectors. See, for example: Lai et al., “Aptamer-Based Electrochemical Detection of Picomolar Platelet-Derived Growth Factor Directly in Blood Serum,” Anal. Chem. 79: 229-233 (2007); Lee et al., “Aptamers and Molecular Recognition Elements for Electrical Nanobiosensors,” Anal. Bioanal Chem. 390: 1023-1032 (2008); So et al., “Single-Walled Carbon Nanotube Biosensors Using Aptamers as Molecular Recognition Elements,” JACS Communications 127: 11906-11907 (2005); and Savran et al., “Micromechanical Detection of Proteins Using Aptamer-Based Receptor Molecules,” Anal. Chem 76:3194-3198 (2004), which are each incorporated herein by reference. An electronic detector can include a recognition element including at least one nucleic acid configured to bind to an analyte. In some embodiments, electronic detector includes piezoelectric sensors. See, for example, Tombelli et al., “Piezoelectric Biosensors: Strategies for Coupling Nucleic Acids to Piezoelectric Devices,” Methods 37: 48-56 (2005), which is incorporated herein by reference. In some embodiments, the electronic detector includes voltammetric sensors. See, for example, Blanco-Lopez et al., “Voltammetric Sensor for Vanillylmanelic Acid Based on Molecularly Imprinted Polymer-Modified Electrodes,” Biosensors and Bioelectronics 18: 352-362 (2003), which is incorporated herein by reference. In some embodiments, the electronic detector includes materials that produce a detectable change when the sensor unit is exposed to an analyte, such as the release of an infrared (IR) detectable dye. See, for example, U.S. Pat. No. 7,964,390 to Rozakis et al., “Sensor System,” which is incorporated herein by reference. In some embodiments, the electronic detector includes graphene-based nanosensors. See, for example, Mannoor et al., “Graphene-based Wireless Bacteria Detection on Tooth Enamel,” Nature Communications, 3:763 doi: 10.1038/ncomms1767 (2012).

The test unit 150 includes an internal channel 1100 in the solid support 200. The internal channel 1100 is not visible in the external view illustrated, but the approximate location of the internal channel 1100 within the solid support 200 is shown with dotted lines in FIG. 11. The internal channel 1100 is connected at a first end to the cavity 210, the internal channel 1100 is connected at a second end to the pathogen detection region 1130. In some embodiments, the internal channel 110 includes a fluid-control film component, the fluid-control film oriented to permit directional flow of fluid from the cavity to the pathogen detection region. See U.S. Pat. No. 6,420,622 to Johnston et al., “Medical Article Having Fluid Control Film,” which is incorporated by reference.

The embodiment of a test unit 150 illustrated in FIG. 11 includes an electronic temperature detector 1110 affixed to the solid support 200. The electronic temperature detector 1110 is affixed to the solid support 200 adjacent to the surface configured to reversibly mate with a skin surface of an individual, or the reverse side of the view shown in FIG. 11. The approximate location of the electronic temperature detector 1110 affixed to the solid support 200 is shown with dotted lines in FIG. 11. The electronic temperature detector 1110 is oriented to detect the temperature of the skin of the individual. The electronic temperature detector 1110 is configured to detect temperatures within a physiological range, i.e. between approximately 96 degrees F. and 105 degrees F.

The embodiment of a test unit 150 illustrated in FIG. 11 includes a processor 1120 attached to both the electronic detector of the pathogen detection region 1130 and the electronic temperature detector 1110. In the embodiment shown, the processor 1120 is attached to the electronic detector of the pathogen detection region 1130 with a wire connector 1160. Although the processor 1120 illustrated in FIG. 11 is illustrated as being attached to the surface of the solid support 200, in some embodiments a processor may be located internally to a solid support, such as between one or more layers of a solid support. The processor 1120 is attached with a wire connector 1170 to a persistent visible indicator 1180. The persistent visible indicator 1120 is configured to initiate a persistent visible indicator in response to a signal from the processor 1120. For example, a visual indicator can include three LED lights of different colors, and the processor can be configured to send a signal to illuminate each color in combination with the results of the look up table. For example, a visual indicator can include LED lights colored blue, green and red. For example, a visual indicator can include a graphics display unit, such as an e-ink device.

The processor is an electronic processor, capable of accepting data, processing data, and sending signals. In some embodiments, the processor includes a wireless transmitter. In some embodiments, the processor includes logic. In some embodiments, the processor includes memory. In some embodiments, the processor is a microprocessor. In some embodiments, the processor includes one or more look-up tables stored in memory. In some embodiments, the processor includes circuitry configured to carry out specific processes as described herein. For example, in some embodiments the processor includes one or more look-up tables including data values received from the electronic temperature detector 1110 and the pathogen detection region 1130, the look-up tables including diagnostic indicators associated with the received data values. For example, it has been shown that evaluation of multiple parameters provides an accurate clinical decision rules for diagnosis of influenza. See, e.g., Ebell et al., “Development and validation of a Clinical Decision Rule for the Diagnosis of Influenza,” JABFM 25 (1) 55-62 (2011), which is incorporated by reference. The processor can include, for example, a look-up table stored in memory, the look-up table including values for information from either or both the pathogen detection region and the temperature detector that are identified as diagnostically “positive” or “negative.” For example, a look-up table may include the temperature detection value of 98.6 degrees F. as “negative” and the temperature detection value of 100 degrees F. as “positive.” For example, a look-up table may include a florescence value above a predetermined background level as “positive,” and a florescence value below a predetermined background level as “negative.” In some embodiments, the processor can include a look up table that incorporates values from both the pathogen detection region and the temperature detector that are identified as diagnostically “positive” or “negative” or “indeterminate.” For example, a combination of temperature detection value of 100 degrees F. and a florescence value above a predetermined background level may be classified as “positive.” For example, a combination of temperature detection value of 98.6 degrees F. and a florescence value below a predetermined background level may be classified as “negative.” For example, a combination of temperature detection value of 100 degrees F. and a florescence value below a predetermined background level may be classified as “indeterminate.”

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer can opt for a mainly hardware and/or firmware vehicle; alternatively, if flexibility is paramount, the implementer can opt for a mainly software implementation; or, yet again alternatively, the implementer can opt for some combination of hardware, software, and/or firmware. Hence, there are several possible vehicles by which the processes and/or devices and/or other technologies described herein can be effected, none of which is inherently superior to the other in that any vehicle to be utilized is a choice dependent upon the context in which the vehicle will be deployed and the specific concerns (e.g., speed, flexibility, or predictability) of the implementer, any of which can vary. Optical aspects of implementations will typically employ optically-oriented hardware, software, and or firmware.

In some implementations described herein, logic and similar implementations can include software or other control structures. Electronic circuitry, for example, can have one or more paths of electrical current constructed and arranged to implement various functions as described herein. In some implementations, one or more media can be configured to bear a device-detectable implementation when such media hold or transmit a device detectable instructions operable to perform as described herein. In some variants, for example, implementations can include an update or modification of existing software or firmware, or of gate arrays or programmable hardware, such as by performing a reception of or a transmission of one or more instructions in relation to one or more operations described herein. Alternatively or additionally, in some variants, an implementation can include special-purpose hardware, software, firmware components, and/or general-purpose components executing or otherwise invoking special-purpose components. Specifications or other implementations can be transmitted by one or more instances of tangible transmission media as described herein, optionally by packet transmission or otherwise by passing through distributed media at various times.

Alternatively or additionally, implementations can include executing a special-purpose instruction sequence or invoking circuitry for enabling, triggering, coordinating, requesting, or otherwise causing one or more occurrences of virtually any functional operations described herein. In some variants, operational or other logical descriptions herein can be expressed as source code and compiled or otherwise invoked as an executable instruction sequence. In some contexts, for example, implementations can be provided, in whole or in part, by source code, such as C++, or other code sequences. In other implementations, source or other code implementation, using commercially available and/or techniques in the art, can be compiled/implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) can be manifested as a Verilog-type hardware description (e.g., via Hardware Description Language (HDL) and/or Very High Speed Integrated Circuit Hardware Descriptor Language (VHDL)) or other circuitry model which can then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, each function and/or operation within such block diagrams, flowcharts, or examples can be implemented, individually and/or collectively, by a wide range of hardware, software, firmware, or virtually any combination thereof. In one embodiment, several portions of the subject matter described herein can be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

In a general sense, the various embodiments described herein can be implemented, individually and/or collectively, by various types of electro-mechanical systems having a wide range of electrical components such as hardware, software, firmware, and/or virtually any combination thereof; and a wide range of components that can impart mechanical force or motion such as rigid bodies, spring or torsional bodies, hydraulics, electro-magnetically actuated devices, and/or virtually any combination thereof. Consequently, as used herein “electro-mechanical system” includes, but is not limited to, electrical circuitry operably coupled with a transducer (e.g., an actuator, a motor, a piezoelectric crystal, a Micro Electro Mechanical System (MEMS), etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs. Examples of electro-mechanical systems include but are not limited to a variety of consumer electronics systems, medical devices, as well as other systems such as motorized transport systems, factory automation systems, security systems, and/or communication/computing systems. Electro-mechanical as used herein is not necessarily limited to a system that has both electrical and mechanical actuation except as context can dictate otherwise.

In a general sense, the various aspects described herein can be implemented, individually and/or collectively, by a wide range of hardware, software, firmware, and/or any combination thereof and can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein can be implemented in an analog or digital fashion or some combination thereof.

At least a portion of the devices and/or processes described herein can be integrated into an image processing system. A typical image processing system generally includes one or more of a system unit housing, a video display device, memory such as volatile or non-volatile memory, processors such as microprocessors or digital signal processors, computational entities such as operating systems, drivers, applications programs, one or more interaction devices (e.g., a touch pad, a touch screen, an antenna, etc.), control systems including feedback loops and control motors (e.g., feedback for sensing lens position and/or velocity; control motors for moving/distorting lenses to give desired focuses). An image processing system can be implemented utilizing suitable commercially available components, such as those typically found in digital still systems and/or digital motion systems. At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system can be implemented utilizing suitable commercially available components, such as those typically found in data computing/communication and/or network computing/communication systems.

The herein described components (e.g., operations), devices, objects, and the discussion accompanying them are used as examples for the sake of conceptual clarity and that various configuration modifications are contemplated. Consequently, as used herein, the specific examples set forth and the accompanying discussion are intended to be representative of their more general classes. In general, use of any specific example is intended to be representative of its class, and the non-inclusion of specific components (e.g., operations), devices, and objects should not be taken limiting.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

With respect to the use of substantially any plural and/or singular terms herein, the plural can be translated to the singular and/or from the singular to the plural as is appropriate to the context and/or application. The various singular/plural permutations are not expressly set forth herein for sake of clarity.

In some instances, one or more components can be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) can generally encompass active-state components and/or inactive-state components and/or standby-state components, unless context requires otherwise.

While particular aspects of the present subject matter described herein have been shown and described, changes and modifications can be made without departing from the subject matter described herein and its broader aspects and, therefore, the appended claims are to encompass within their scope all such changes and modifications as are within the true spirit and scope of the subject matter described herein. Terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). Typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, the recited operations therein can generally be performed in any order. Also, although various operational flows are presented in a sequence(s), it should be understood that the various operations can be performed in other orders than those which are illustrated, or can be performed concurrently. Examples of such alternate orderings can include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

### PROPHETIC EXAMPLES

The below prophetic examples are included as an aid to understanding, and are not meant to be limiting.

### Example 1

A low-cost, multifunctional test unit to measure body temperature and to detect influenza virus infections.

A test unit is fabricated from a plastic-coated paper material with a plurality of layers forming a solid support. The solid support is substantially planar, between approximately 1-3 millimeter (mm) in thickness and includes a surface of approximately 3 centimeters (cm) by 9 cm in size. The solid support is flexible, and reversibly mates with the surface of an individual's skin when placed adjacent to the skin, such as the forehead or the underside of the wrist. The solid support is fabricated with a series of indentations and channels, as described further below.

The test unit incorporates a persistent chemical thermometer which indicates body temperature after contact with the skin. A persistent chemical thermometer is constructed by creating indentations in the solid support which are subsequently filled with chemical temperature indicators to form indicator wells. Multiple indentations are constructed in the solid support and filled with chemical temperature indicators composed of ortho-bromonitrobenzene (OBNB) and ortho-chloronitrobenzene (OCNB) combined at various ratios, each ratio specific to a temperature in a gradient of indicator wells. Ratios of OBNB:OCNB varying between approximately 56.2:43.8 and 96.0:4.0 are used to create persistent temperature indicators that change from solid to liquid phase at temperatures ranging from 96.0° F. to 105.0° F. See e.g., U.S. Pat. Nos. 4,232,552, 4,339,207 and 4,362,645, each titled “Temperature Indicating Compositions of Matter,” to Hof et al., which are each incorporated herein by reference.

During use, the patient's temperature is indicated by the color of the indicator wells. Indicator wells which reach their specific phase transition temperature change their appearance and are persistent, i.e., they remain liquid even after the device is removed from the skin and the indicator wells cool. See e.g., U.S. Pat. Nos. 4,232,552, 4,339,207 and 4,362,645, each titled “Temperature Indicating Compositions of Matter,” to Hof et al., which are each incorporated herein by reference. The visible color of the indicator wells are a visible record of the patient's temperature which is persistent on the test strip after it has been removed from an individual's skin. Depending on the configuration of the indicator wells, the visible color relative to the temperature can be persistent for at least several minutes and up to several hours. See e.g., U.S. Pat. Nos. 4,232,552, 4,339,207 and 4,362,645, each titled “Temperature Indicating Compositions of Matter,” to Hof et al., which are each incorporated herein by reference.

The test unit also contains analyte detection chambers which will function to detect influenza virus in nasal fluid from the patient and indicate the detection. Nasal fluid is collected in a cavity or indentation in one end of the test strip. The collection cavity is connected via a channel, which is interior to the solid support, to a reagent chamber within the test unit. The reagent chamber is also connected by interior channels to detection chambers. See, e.g. FIG. 6. Nasal fluid or nasopharyngeal fluid will be obtained by aspiration, which is then applied to the cavity on the test strip. The fluid then will move into the reagent chamber within the test strip via the interior channel through capillary action.

The reaction chamber will contain one or more detection reagents configured to bind to pathogen proteins present in the nasal or nasopharyngeal fluid. For example, detection reagents will include monoclonal antibodies (MAbs) conjugated with colloidal gold particles which recognize an influenza viral protein, NS1, and are used to detect viral antigen in a lateral flow assay. See U.S. Patent Application Publication No. 2012/0184462, “Lateral Flow Assay Using Two Dimensional Features,” to O'Farrell and Tisone, which is incorporated by reference. Gold-conjugated MAbs that recognize NS1 protein from all known strains of influenza type A or influenza type B are combined with the nasal or nasopharyngeal fluid sample. After combining, the MAbs will specifically bind to any influenza NS1 proteins present in the sample. More information regarding MAb construction, nasal fluid collection, assay details and assay sensitivity may be found in: U.S. Pat. No. 8,163,474, “NS1-NP Diagnostics of Influenza Virus Infection,” to Lu et al.; and U.S. Patent Application Publication No. 2010/0143884, “Detection of Influenza Virus,” to Lu et al., which are each incorporated herein by reference. For example, nasal or nasopharyngeal fluid can be combined with gold-conjugated pan-influenza A and B MAbs in a total volume of approximately 200 microliters.

After the combination of the nasal or nasopharyngeal fluid and the detection reagents in the reaction chamber, the sample is directed via a channel to an analyte detection chamber within the test unit. The sample flows by capillary action through a membrane (e.g., nitrocellulose) with capture MAbs immobilized at different locations, or spots in the membrane. For example, MAbs specific for influenza A, influenza A subtypes (e.g., H1N1, H3N2, H3N1, H5N1) and influenza B can be immobilized on a nitrocellulose membrane in a pixilated pattern that allows independent flow, capture and detection of viral proteins at each point in the pattern. MAbs in solution at approximately 0.5 mg/ml can be immobilized in a pattern which is labeled to identify the locations and specificities of the capture MAbs. Lateral flow assays to detect viral antigens in multiplex are described. See: U.S. Pat. No. 8,163,474, “NS1-NP Diagnostics of Influenza Virus Infection,” to Lu et al.; U.S. Patent Application Publication No. 2012/0184462, “Lateral Flow Assays Using Two Dimensional Features,” to O'Farrell et al.; and U.S. Patent Application Publication No. 2010/0143884, “Detection of Influenza Virus,” to Lu et al., which are each incorporated herein by reference. Osmotic flow and capture of influenza antigens bound to gold-conjugated MAbs occurs within minutes and the results of the lateral flow assay are obtained by visual examination of the matrix for accumulation of colloidal gold over specific capture MAbs. For example, a type A influenza virus, H1N1, may show gold deposition over two capture MAbs, a pan-influenza A MAb and a H1N1-specific MAb. These appear as colored dots on the test unit.

### Example 2

A multifunctional test unit is used to measure body temperature, detect viral infections and provide a preliminary screening tool for caregivers of nursing home residents.

In response to a reported seasonal epidemic of influenza, residents of a nursing home are screened daily by caregivers with the disposable multifunctional test unit, as described in Example 1. The results indicate potentially infected individuals for further evaluation. The results can assist caregivers to determine what influenza viral subtype(s) may be present, potentially indicating routes of infection within the nursing home. Combined measurement of temperature and detection of viral antigens allows healthcare workers to rapidly screen residents who would benefit from further evaluation. See: “CDC—Seasonal Influenza (flu)—Rapid Diagnostic testing for Influenza: Information for Clinical Laboratory Directors,” downloaded on Oct. 9, 2012, which is incorporated by reference.

Body temperature is an informative physiological parameter to indicate influenza infection, especially in older adults, ages 55-80. See, e.g., Woolpert et al., “Determination of Clinical and Demographic Predictors of Laboratory-Confirmed Influenza with Subtype Analysis,” BMC Infectious Diseases 12: 129, (2012) and Michiels et al., “Clinical Predication Rules Combining Signs, Symptoms and Epidemiological Context to Distinguish Influenza from Influenza-like Illnesses in Primary Care: A Cross Sectional Study,” BMC Family Practice 12:4, (2011), which are each incorporated herein by reference. Body temperature measurements predict influenza with high sensitivity, but display low specificity (i.e., a high number of false positives, see e.g., Michiels et al., Ibid.). In the test unit, temperature measurements are complemented by rapid immunoassays for viral antigens which display high specificity and low sensitivity (see e.g., Chartrand et al., “Accuracy of Rapid Influenza Diagnostic Tests” Ann. Intern. Med. 156: 500-511, (2012) and Gavin et al., “Review of Rapid Diagnostic Tests for Influenza,” Clin. Appl. Immunol. Rev. 4: 151-172, (2003), which are each incorporated herein by reference). The combination of data regarding both temperature and influenza antigens provides a rapid screening tool for use by caregivers to prioritize the needs of residents.

For example, a first patient is tested with a first test unit. A temperature ≧100° F. is indicated with the persistent chemical indicator of temperature in the first test unit. The patient's nasal fluid is tested in the lateral flow immunoassay on the test unit and specific spots on the test unit darken. The caregiver administering the test unit can place the first patient's test unit in a location for subsequent evaluation by a trained medical professional, such as a nurse, while continuing to test further residents with additional test units. A photo of the first test unit can be maintained in the patient's medical record for further review as needed, or to support a medical record or history.

When the first patient's test unit is evaluated, it is found to test positive for influenza, specifically influenza A and H1N1. The evaluating medical professional can read the temperature of the patient by examining the color of the indicator wells. The evaluating medical professional can compare the spots or pattern on the test unit with one or more reference pictures to evaluate the immunoassays on the test unit. The combination of fever (temperature ≧100° F.) and the immunoassay result is consistent with an ongoing infection. Elevated temperature plus detection of viral antigens suggest strongly that the patient is infected with influenza. The caregiver team at the nursing facility can then initiate medical care for the first patient, as well as infection control procedures. For example, the patient may be told to minimize contacts with other patients or he/she may be confined to their room to reduce the likelihood of transmission of influenza. The patient may be treated initially with therapeutics or prophylactics. If desired by the medical team, the patient may also be retested with a lengthier, “gold standard” test, e.g., RT-PCR or in vitro culture methods to verify the preliminary diagnosis.

A second patient is screened by a caregiver with a second test unit. On evaluation, the second test unit displays an elevated temperature, ≧100° F., but the nasal fluid tests negative for influenza antigens in the lateral flow assay. A preliminary diagnosis of “possible infection” for the second patient is recorded by the caregiver, as indicated by the test unit (see e.g., Woolpert et al., “Determination of Clinical and Demographic Predictors of Laboratory-Confirmed Influenza with Subtype Analysis,” BMC Infectious Diseases 12: 129, (2012), which is incorporated by reference). Subsequently, as desired by the medical team, the patient may be referred for testing with a “gold standard” test or simply told to limit contact with other patients and retested with the multifunction test unit the following day. In addition, the second patient may be treated with antiviral drugs to limit any viral infection and shorten the duration of disease (see e.g.: Moscona, “Neuraminidase Inhibitors for Influenza”, New Engl. J. Med. 353: 1363-1373, (2005); and “CDC-Treating Influenza” dated Jul. 12, 2012, which are incorporated by reference).

A third patient is tested with the multifunctional test unit. On evaluation of the third test unit, it does not display an elevated temperature (i.e. fever), but the nasal fluid tests positive for influenza A viral antigen in the lateral flow immunoassay. The patient is given a preliminary diagnosis of “tentative positive for influenza infection.” The patient may be retested to verify the results and to determine the subtype of influenza virus by using other tests, e.g., RT-PCR. The patient may also be segregated from other patients to minimize the potential spread of infection, or otherwise asked to take precautions to minimize the spread of infection (i.e. wear a face mask in the recreation area of the nursing home). If the medical team chooses, the patient may be administered antiviral drugs as a prophylaxis.

Daily test results from the multifunctional test unit may be documented by scanning test units into each patient's electronic medical record. The cumulative test results may be used to track the course of a patient's disease, to monitor the prevalence of influenza in the nursing home, to provide information regarding the spread of disease, and to segregate or confine the patients during an epidemic.

### Example 3

A multifunctional oral test unit device to detect fever, inflammation and microbial infections.

An oral-use test unit is constructed including a thermometer and analyte immunoassay detection sections to detect markers of infection. The test unit is constructed from a polypropylene solid support with separate sections containing a chemical thermometer and analyte detection chambers.

The chemical thermometer includes individual wells which hold temperature indicator chemicals and a transparent cover. For example, the wells may be fabricated from indentations in the polypropylene solid support and covered with a transparent film cover after they are filled. See e.g., U.S. Pat. Nos. 4,232,552, 4,339,207 and 4,362,645, each titled “Temperature Indicating Compositions of Matter,” to Hof et al., which are each incorporated herein by reference. For example, 45 multiple wells can be constructed and filled with temperature indicators composed of ortho-bromonitrobenzene (OBNB) and ortho-chloronitrobenzene (OCNB) combined at various ratios. Ratios of OBNB:OCNB varying between approximately 56.2:43.8 and 96.0:4.0 can be used to create indicators that change from solid to liquid phase at temperatures ranging from 96.0° F. to 105.0° F. See e.g., U.S. Pat. Nos. 4,232,552, 4,339,207 and 4,362,645, each titled “Temperature Indicating Compositions of Matter,” to Hof et al., which are each incorporated herein by reference. Mixtures of solid OBNB and OCNB at various ratios are imprinted in the wells and labeled with their corresponding phase transition temperatures. Persistent chemical thermometers indicate the temperature by a visual change in appearance when going from solid to liquid, or by including dyes in the indicator chemicals which become visible after a phase change occurs. Chemical thermometers adapted for measuring temperature with an accuracy of approximately 0.2° F. are described. See e.g., U.S. Pat. Nos. 4,232,552, 4,339,207 and 4,362,645, each titled “Temperature Indicating Compositions of Matter,” to Hof et al., which are each incorporated herein by reference. The visible color of the indicator wells are a visible record of the patient's temperature which is persistent on the test strip after it has been removed from an individual's oral cavity. Depending on the configuration of the indicator wells, the visible color relative to the temperature can be persistent for at least several minutes and up to several hours. See e.g., U.S. Pat. Nos. 4,232,552, 4,339,207 and 4,362,645, each titled “Temperature Indicating Compositions of Matter,” to Hof et al., which are each incorporated herein by reference.

Thin polypropylene used to form the solid support and a transparent cover over the thermometer wells allows efficient transfer of heat to the indicator wells from the mouth. The patient's temperature is read after placing the unit test device under the patient's tongue for approximately one minute and then removing the test unit to examine the indicator wells which are visible through transparent film covers. Indicator wells that reach their phase transition temperature, change their appearance and indicate the patient's temperature provide a record of the temperature which may be stable for at least several minutes and up to several hours. The temperature indicator chemicals are persistent, i.e., they main remain liquid even after the device is removed from the mouth and the indicator wells cool below their melting points. Thus, a visible record of the patient's temperature, as a set of indicator wells with or without a phase change, remains on the device for at least several minutes and up to several hours after removal of the test unit from the mouth. This allows a first caregiver to administer the test and then continue administration of tests to other individuals, with the evaluation to be done by a follow-up caregiver within a short time period thereafter.

The multifunction test unit also has a section configured for analyte detection which detects inflammatory markers and microbial pathogens which are present in saliva and oral mucosal exudate. Test systems for detection of oral analytes are described (see e.g., U.S. Pat. No. 7,700,305, “Analyte Detection”, to Toranto, which is incorporated herein by reference). The solid support of the multifunction test unit has an indentation connected to a reservoir. The indentation is positioned to collect saliva when the device is placed in the mouth of the patient. The reservoir can hold 0.1 to 1.0 mL of saliva, and is connected by a channel to a reaction chamber within the solid support of the test unit. The reaction chamber of the test unit contains detection MAbs for inflammatory markers and microbial pathogens. The detection MAbs are labeled with colloidal gold, and can be visually detected in a lateral flow assay (see e.g., U.S. Patent Application Publication No. 2012/0184462, “Lateral Flow Assays Using Two Dimensional Features,” to O'Farrell et al., which is incorporated herein by reference). Channels emanating from the reaction chamber distribute the analytes with bound detection MAbs to an analyte detection chamber which contains a nitrocellulose membrane with capture MAbs immobilized on the membrane surface. The sample flows via capillary action through the nitrocellulose membrane with capture MAbs immobilized at labeled locations. The MAbs are immobilized on the nitrocellulose membrane in a pixilated pattern that allows independent flow, capture and detection of antigens at each point in the pattern. Lateral flow assays to detect antigens in multiplex are described (see e.g., U.S. Patent Application Publication No. 2012/0184462, “Lateral Flow Assays Using Two Dimensional Features,” to O'Farrell et al., and U.S. Patent Application Publication No. 2010/0143884, “Detection of Influenza Virus,” to Lu et al., which are each incorporated herein by reference). The lateral flow assays can be evaluated by visual inspection of the spot pattern on the lateral flow assay section of the test unit.

Detection MAbs and capture MAbs which recognize inflammatory markers can be used to identify a broad spectrum of patients with different microbial infections. For example, detection of C-reactive protein (CRP), interleukin 1-β, and β-glucuronidase in saliva can indicate an ongoing microbial infection derived from viral and bacterial sources. See e.g.: Miller et al., “Current Developments in Salivary Diagnostics”, Biomark Med. 4:171-189, (2010); Watson et al., “Raised Inflammatory Markers”, BMJ 344:e454, (2012), (doi: 10.1136/bmj.e454); and Mogensen, “Pathogen Recognition and Inflammatory Signaling in Innate Immune Defenses,” Clin. Microbiol. Rev. 22: 240-273, (2009) which are each incorporated herein by reference.

To test for specific microbial pathogens, the test unit device can include antibodies specific for microbial pathogens (e.g., influenza virus, respiratory syncytial virus, adenovirus, parainfluenza virus, Streptococcus pneumoniae, Neisseria meningitidis and Mycoplasma). Antibodies specific for inflammatory markers and microbial pathogens can be obtained, for example, from KPL, Inc., (Gaithersburg, Md.). Detection and capture antibodies for microbial pathogens can identify pathogens associated with any infectious fever and/or inflammation detected by the test unit. Multi-parameter test units, which determine a physical parameter, e.g., body temperature, assess inflammation markers and also detect microbial pathogens can be used by a caregiver team to improve the accuracy of initial diagnosis (see e.g., Watson et al., “Raised Inflammatory Markers”, BMJ 344:e454, (2012), (doi: 10.1136/bmj.e454), which is incorporated by reference).

### Example 4

Use of an oral test unit device to provide a preliminary diagnosis for patients at a rural clinic.

A small rural clinic that serves a large number of farm workers and laborers from the surrounding area uses an inexpensive multifunctional oral test unit to make a preliminary diagnosis for patients at the clinic and recommend further care. Patients complaining of fever, headache and other symptoms of microbial infections (e.g., bacterial and viral respiratory infections) are tested with an oral test unit provided to them in the waiting room of the clinic prior to seeing a healthcare professional.

The patients are each given a multifunctional oral test unit (e.g., see Example 3) with persistent indicators of body temperature and pathogen immunoassays when they check in to the clinic, and they are instructed to remove a protective cover from the device and place it in their mouth for approximately two minutes. They are asked to return the test unit device to the front desk after the two minutes has elapsed. Subsequently, the returned test units are evaluated by a healthcare worker. The evaluating healthcare worker can attach each patient's test unit to their chart, or add a photo or scan of the test unit to a digital medical record for the patient. The evaluating healthcare worker can report a preliminary diagnosis of infection, if indicated by the test unit, and make recommendations for care of the patients.

For example, a first patient's test unit indicates that he has a temperature of approximately 100° F. and that his saliva is positive for an inflammatory marker, C-Reactive Protein (CRP). Elevated temperature and an inflammatory marker suggest the patient may have a microbial infection (see e.g., Woolpert et al., “Determination of Clinical and Demographic Predictors of Laboratory-Confirmed Influenza with Subtype Analysis,” BMC Infectious Diseases 12: 129, (2012) and Watson et al., “Raised Inflammatory Markers”, BMJ 344:e454, (2012), (doi: 10.1136/bmj.e454), which are each incorporated by reference). The test unit also detects a specific microbial pathogen in the patient's saliva, Streptococcus pneumoniae. Based on the combination of physical (temperature), inflammation (CRP) and pathogen data, the evaluating health care worker finds that the test unit results indicate a preliminary diagnosis of a bacterial (Streptococcus pneumoniae) infection. The evaluating health care worker recommends the patient be seen by a physician or nurse practitioner at that visit to the clinic. The physician or nurse practitioner can perform a clinical exam on the first patient to confirm the preliminary diagnosis of a bacterial infection and/or rely on the test unit data to treat the patient (e.g., prescribe an antibiotic).

A second patient reports a cough to the health care worker undertaking the initial screen. The oral-use test unit from the second patient indicates she has a temperature of approximately 100° F., but her saliva does not test positive for markers of inflammation. Also, data from the oral-use test unit does not detect a specific microbial pathogen in her saliva. Results from the test unit indicate to the evaluating healthcare worker that the second patient should be given a preliminary diagnosis of “uninfected” despite a slightly elevated temperature. The healthcare worker, consequently, may not recommend the patient see a physician or nurse practitioner, but instead they may recommend home care and vigilance for any symptoms that may arise. The patient is encouraged to return to the clinic, and be retested with another test unit, if symptoms persist or worsen. A photo or scan of the first test unit can be added to the second patient's medical record for use as a baseline for comparison with subsequent test units.

In addition, data from a plurality of test units used over time can assist the clinic to evaluate infections within groups or within the community for public health assessment. For example, if data from a plurality of test units indicate that multiple workers from a particular work group have tested positive for the same pathogen within a particular time period (e.g. a week, or two weeks), community public health workers may choose to follow-up with the employer or work group to advise them of infection control procedures (e.g. effective handwashing protocols).

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A test unit, comprising:
a solid support including a surface configured to reversibly mate with a skin surface of an individual;
a cavity in the solid support;
a reaction chamber internal to the solid support, the reaction chamber including a plurality of detection agents within a space;
a first internal channel in the solid support, the first internal channel connected at a first end to the cavity, the first internal channel connected at a second end to the reaction chamber;
at least one pathogen detection region internal to the solid support, each of the at least one pathogen detection region including a visible indicator of one or more of the plurality of detection agents;
a second internal channel in the solid support, the second internal channel connected at a first end to the reaction chamber, the second internal channel connected at a second end to the at least one pathogen detection region;
a temperature detector affixed to the solid support; and
a persistent visible temperature indicator attached to the temperature detector.

2. The test unit of claim 1, wherein the solid support including the surface configured to reversibly mate with the skin surface of an individual comprises:
a substantially flat solid support.

3. (canceled)

4. The test unit of claim 1, wherein the solid support including the surface configured to reversibly mate with the skin surface of an individual comprises:
a plurality of layers combined to form a structure of the solid support.

5. The test unit of claim 1, wherein solid support including the surface configured to reversibly mate with the skin surface of an individual comprises:
a surface configured for placement adjacent to the skin surface; and
bio-compatible adhesive on the surface configured for placement adjacent to the skin.

6-9. (canceled)

10. The test unit of claim 1, wherein the reaction chamber internal to the solid support, the reaction chamber including the plurality of detection agents within the space comprises:
one or more detection agents including an antibody configured to bind with a pathogen protein.

11. The test unit of claim 1, wherein the reaction chamber internal to the solid support, the reaction chamber including the plurality of detection agents within the space comprises:
one or more detection agents including an enzyme.

12. (canceled)

13. The test unit of claim 1, wherein the at least one pathogen detection region internal to the solid support comprises:
a plurality of pathogen detection regions.

14. (canceled)

15. The test unit of claim 1, wherein the persistent visible temperature indicator attached to the temperature detector comprises:
a persistent chemical indicator.

16. The test unit of claim 1, wherein the persistent visible temperature indicator attached to the temperature detector comprises:
a persistent electronic indicator.

17-18. (canceled)

19. The test unit of claim 1, further comprising:
a sensor of a physiological condition affixed to the solid support; and
an indicator attached to the sensor of the physiological condition.

20. The test unit of claim 19, wherein the sensor of a physiological condition comprises:
a sensor of a sweat level on the skin surface of the individual at a location adjacent to the solid support.

21. The test unit of claim 1, further comprising:
a processor attached to both the at least one pathogen detection region internal to the solid support and to the temperature detector affixed to the solid support, the processor configured to accept information; and
a visual indicator attached to the processor.

22. A test unit, comprising:
a solid support including a surface configured to reversibly mate with a skin surface of an individual;
a nasal fluid receiving cavity in the solid support;
a reaction chamber internal to the solid support, the reaction chamber including a plurality of detection agents within a space;
a first internal channel to the solid support, the first internal channel including a first end attached to the nasal fluid receiving cavity, the first internal channel including a second end attached to the reaction chamber;
at least one pathogen detection region internal to the solid support, each of the at least one pathogen detection region including a visible indicator of one or more of the plurality of detection agents;
at least one second internal channel to the solid support, the at least one second internal channel including a first end attached to the reaction chamber, the at least one second internal channel including a second end attached to the at least one pathogen detection region;
a temperature detector affixed to the solid support; and
a persistent visible temperature indicator attached to the temperature detector.

23. The test unit of claim 22, wherein the solid support including the surface configured to reversibly mate with the skin surface of the individual comprises:
a substantially flat solid support.

24. (canceled)

25. The test unit of claim 22, wherein the solid support including the surface configured to reversibly mate with the skin surface of the individual comprises:
a plurality of layers combined to form a structure of the solid support.

26. The test unit of claim 22, wherein the solid support the surface configured to reversibly mate with the skin surface of the individual comprises:
a surface configured for placement adjacent to the skin surface; and
bio-compatible adhesive on the surface configured for placement adjacent to the skin surface.

27-29. (canceled)

30. The test unit of claim 22, wherein the reaction chamber internal to the solid support, the reaction chamber including the plurality of detection agents within the space comprises:
one or more detection agents including an antibody configured to bind with a pathogen protein.

31. The test unit of claim 22, wherein the reaction chamber internal to the solid support, the reaction chamber including the plurality of detection agents within the space comprises:
one or more detection agents including an enzyme.

32. (canceled)

33. The test unit of claim 22, wherein the at least one pathogen detection region internal to the solid support comprises:
a plurality of pathogen detection regions.

34. (canceled)

35. The test unit of claim 22, wherein the temperature detector affixed to the solid support comprises:
a chemical-based temperature detector.

36. (canceled)

37. The test unit of claim 22, wherein the temperature detector affixed to the solid support comprises:
an electronic temperature detector.

38-39. (canceled)

40. The test unit of claim 22, further comprising:
a sensor of a physiological condition of the individual affixed to the solid support; and
an indicator attached to the sensor of the physiological condition of the individual.

41. The test unit of claim 40, wherein the sensor of the physiological condition comprises:
a sensor of a sweat level on the skin surface of the individual at a location adjacent to the solid support.

42. The test unit of claim 22, further comprising:
a processor attached to both the at least one pathogen detection region internal to the solid support and to the temperature detector affixed to the solid support, the processor configured to accept information; and
a visual indicator attached to the processor.

43. A test unit, comprising:
a solid support including a region configured to be enclosed within an oral cavity of an individual;
an oral fluid receiving cavity in the solid support;
a reaction chamber internal to the solid support, the reaction chamber including a plurality of detection agents within a space;
a first internal channel to the solid support, the first internal channel including a first end attached to the oral fluid receiving cavity, the first internal channel including a second end attached to the reaction chamber;
at least one pathogen detection region internal to the solid support, each of the at least one pathogen detection region including a visible indicator of one or more of the plurality of detection agents;
at least one second internal channel to the solid support, the at least one second internal channel including a first end attached to the reaction chamber, the at least one second internal channel including a second end attached to the at least one pathogen detection region;
a temperature detector affixed to the solid support; and
a persistent visible temperature indicator attached to the temperature detector.

44-46. (canceled)

47. The test unit of claim 43, wherein the solid support including the region configured to be enclosed within the oral cavity of the individual comprises:
one or more conduits within a structure of the solid support.

48. The test unit of claim 43, wherein the solid support including the region configured to be enclosed within the oral cavity of the individual comprises:
a first region configured to fit within the oral cavity; and
a second region configured to be positioned outside the oral cavity, wherein the persistent visible temperature indicator and the at least one pathogen detection region are affixed to the second region.

49-51. (canceled)

52. The test unit of claim 43, wherein the reaction chamber internal to the solid support comprises:
one or more detection agents including an antibody configured to bind with a pathogen protein.

53. The test unit of claim 43, wherein the reaction chamber internal to the solid support comprises:
one or more detection agents including an enzyme.

54-55. (canceled)

56. The test unit of claim 43, wherein the at least one pathogen detection region comprises:
a plurality of pathogen detection regions.

57. (canceled)

58. The test unit of claim 43, wherein the temperature detector affixed to the solid support comprises:
a chemical-based temperature detector.

59. The test unit of claim 43, wherein the temperature detector affixed to the solid support comprises:
an electronic temperature detector.

60. The test unit of claim 43, wherein the persistent visible temperature indicator attached to the temperature detector comprises:
a persistent chemical indicator.

61. The test unit of claim 43, wherein the persistent visible temperature indicator attached to the temperature detector comprises:
a persistent electronic indicator.

62-63. (canceled)

64. The test unit of claim 43, further comprising:
a sensor of a physiological condition within the oral cavity, the sensor affixed to the solid support at the region configured to be enclosed within the oral cavity of the individual.

65. The test unit of claim 43, further comprising:
a processor attached to both the at least one pathogen detection region internal to the solid support and to the temperature detector affixed to the solid support, the processor configured to accept information; and
a visual indicator attached to the processor.

66. A test unit, comprising:
a solid support including a surface configured to reversibly mate with a skin surface of an individual;
a cavity in the solid support;
at least one pathogen detection region internal to the solid support, each of the at least one pathogen detection region including an electronic detector of at least one analyte;
an internal channel in the solid support, the internal channel connected at a first end to the cavity, the internal channel connected at a second end to the at least one pathogen detection region;
an electronic temperature detector affixed to the solid support;
a processor attached to both the electronic detector and the electronic temperature detector; and
a persistent visible indicator attached to the processor.

67. The test unit of claim 66, further comprising:
a reaction chamber internal to the solid support, the reaction chamber including a plurality of detection agents within a space, the reaction chamber attached to the internal conduit, the plurality of detection agents detectable by the electronic detector.
